(19) United States
(12) Patent Application Publication
(10) Pub. No.: US 2021/0186870 A1
(43) Pub. Date: Jun. 24, 2021

(54) IMPROVED CANNABINOID BIOAVAILABILITY

(71) Applicant: EMERALD HEALTH THERAPEUTICS CANADA INC., Vancouver (CA)

(72) Inventors: Freydoun GARABAGI, Vancouver (CA); Gaetano MORELLO, Vancouver (CA); Christopher WAGNER, Vancouver (CA)

(21) Appl. No.: 17/271,146

(22) PCT Filed: Aug. 26, 2019
(86) PCT No.: PCT/IB2019/000964

§ 371 Date: Feb. 24, 2021

Related U.S. Application Data
(60) Provisional application No. 62/723,229, filed on Aug. 27, 2018.

(51) Int. Cl. A61K 9/00; A61K 31/352; A61K 31/05; A61K 31/353; A61K 47/44; A61K 9/48; A61K 9/20; A61K 9/107; A61K 31/192; A61J 1/03; A61K 47/26; A61P 25/22; A61P 25/04; A61P 25/00; A61P 25/26; A61P 3/04; A61P 3/10; A23L 33/105; A23L 33/115; A23L 33/00

(52) U.S. Cl. CPC A61K 9/0053; A23V 2002/00; A61K 31/05; A61K 31/353; A61K 47/44; A61K 9/4875; A61K 9/2086; A61K 9/1075; A61K 31/192; A61J 1/035; A61K 47/26; A61P 25/22; A61P 25/04; A61P 25/00; A61P 25/26; A61P 3/04; A61P 3/10; A61K 9/2068; A61K 9/2018; A23L 33/105; A23L 33/115; A23L 33/40; A61K 31/352

(57) ABSTRACT

Described herein are cannabinoid formulations for oral administration. Further described herein are methods for orally administering one or more cannabinoids to a subject in need thereof.

### CROSS-REFERENCE TO RELATED APPLICATIONS

This application claims the benefit of U.S. Provisional Application No. 62/723,229, filed Aug. 27, 2018, which is incorporated herein by reference in its entirety.

### BACKGROUND OF THE INVENTION

Cannabinoid molecules, such as tetrahydrocannabinol (THC), cannabidiol (CBD) cannabigerol (CBG), tetrahydrocannabinolic acid (THCA), cannabidiolic acid (CBDA), cannabinchromene (CBC), and cannabigerolic acid (CBGA), are known to cause a variety of therapeutic and psychotropic effects when administered to a subject.

Cannabinoids from the plant genus Cannabis could be considered a type of natural health product, but historically they have not been legally available. The laws which have criminalized possession or use of cannabis have been the primary restraint. These laws were put in place apparently to control the use of one specific cannabinoid, delta-9 tetrahydrocannabinol (THC), which causes a mild temporary psychotropic effect in users. But it is well known that dozens of other cannabinoids are also present in cannabis, none of which have psychotropic effects, and which have, or potentially may have, beneficial pharmacological effects in humans. These alternate cannabinoids which are devoid of psychotropic effect include but are not limited to tetrahydrocannabinolic acid (THCA), cannabinolic acid (CBNA), cannabidiolic acid (CBDA) and cannabigerolic acid (CBGA) and the de-carboxylated derivatives cannabinol (CBN), cannabidiol (CBD) and cannabigerol (CBG). With the de-criminalization of cannabis in some jurisdictions, the opportunity for use of cannabinoids in diverse health regimens is becoming possible.

Cannabinoids are key players in biological systems and bind to receptors in the body known as cannabinoid receptors which have been implicated in a variety of physiological functions, including appetite, pain, emotional behavior (mood), memory, and inflammation. There are currently two known well defined subtypes of cannabinoid receptors, called CB1 and CB2. The CB1 receptor (CB1R) is expressed mainly in the brain (central nervous system or “CNS”), and also in the lungs, liver and kidneys. The CB2 receptor (CB2R) is expressed mainly in the immune system and in hematopoietic or blood cells. There is mounting evidence that there are other novel cannabinoid receptors which are similar to CB1R and CB2R but which are unique.

In general, cannabinoid molecules have traditionally been administered via inhalation, for example by smoking or heating and inhaling the vapours of cannabis plants or cannabis extracts. Oral administration of such molecules, as compared with administration via inhalation, can provide improved consistency of dosing, more discreet administration, and increased convenience for users.

### SUMMARY OF THE INVENTION

Described herein are cannabinoid formulations for oral administration, providing consistency of dosing and discreet administration.

In one aspect, described herein are cannabinoid formulations for oral administration, and the oral administration thereof, the formulations comprising: one or more cannabinoids selected from the group consisting of: 0.1-100 mg tetrahydrocannabinol (THC); 0.1-750 mg tetrahydrocannabinolic acid (THCA); 0.1-750 mg cannabidiol (CBD); 0.1-750 mg cannabidiolic acid (CBDA); 0.1-750 mg cannabigerol (CBG); 0.1-750 mg cannabinchromene; and a lipid carrier comprising one or more lipids selected from the group consisting of: camelina oil; a marine phospholipid; a krill oil; a fish oil; chia seed oil; flaxseed oil; an oil comprising an omeg-3 to omega-6 ratio of about 1.0 or higher, about 1.5 or higher, about 2.0 or higher, or about 2.2. or higher; a hydrolyzed oil; a vegetable oil; hemp oil; an oil containing medium-chain fatty acids (MCFAs); and an oil containing long-chain fatty acids (LCFAs). In some embodiments, the lipid carrier comprises or consists of camelina oil. In some embodiments, one or more of the one or more cannabinoids is dissolved and/or suspended in the lipid carrier. In some embodiments, the cannabinoid formulation is in a unit dosage form selected from a pill, tablet, capsule, film, wafer, lollipop, lozenge, oil, tincture, or syrup. In some embodiments, the cannabinoid formulation is an orally disintegrating pill, tablet, capsule, film, or wafer. In some embodiments, the cannabinoid formulation is a pill or tablet and further comprises an enteric coating for containing the one or more cannabinoids and the lipid carrier. In some embodiments, the cannabinoid formulation is a pill, tablet, or capsule and further comprises an outer shell that is substantially opaque to one or both of ultraviolet and visible light. In some embodiments, the cannabinoid formulation further comprises a further carrier oil. In some embodiments, the cannabinoid formulation further comprises a stabilizer. In some embodiments of the cannabinoid formulation, the lipid carrier is present in the form of an emulsion. In some embodiments of the cannabinoid formulation, the emulsion is a nanoemulsion.

In some embodiments of the cannabinoid formulation, upon administration of the cannabinoid formulation to a subject, a target bioavailability is achieved. In some embodiments, the formulation comprises a first unit dose amount of a dosed cannabinoid, further wherein the administration is by oral administration, and wherein the target bioavailability is a bioavailability of THC or a THC liver metabolite which is greater than a baseline bioavailability, the baseline bioavailability being that achievable from oral administration to the subject of a control composition containing the first unit dose amount of the dosed cannabinoid in a carrier comprising at least 90%, or consisting of, sesame oil. In some embodiments, the dosed cannabinoid is THC and wherein the cannabinoid formulation comprises about 30-50% of the unit dose amount of THC in an emulsion. In some embodiments, the target bioavailability is a THC or THC liver metabolite bioavailability in the subject that is at least 140%, as measured by subject Cmax and/or subject AUC, of the baseline bioavailability. In some embodiments, the target bioavailability is a THC or THC liver metabolite bioavailability in the subject that is: (i) at least 108.5%, at least 146.9%, at least 159.6%, or at least 200%, as measured by subject Cmax, and/or (ii) at least 144.3%; at least 159.6%, at least 200%, or at least 252.1%, as measured by subject AUC, of the baseline bioavailability. In some embodiments, the target bioavailability is a bioavailability of: THC; 11-OH-THC; and/or THC—COOH.

In some embodiments of the cannabinoid formulation, one or more of the cannabinoids is in the form of an organic solvent-based extract of cannabis. In some embodiments of the cannabinoid formulation, at least one further cannabinoid selected from the group consisting of cannabigerolic acid (CBGA), tetrahydrocannabivarin(THCV). In some embodiments, the cannabinoid formulation comprises CBD in an amount between 10-50 mg. In some embodiments, the cannabinoid formulation comprises 25 mg CBD. In some embodiments, the cannabinoid formulation comprises 500 mg CBD. In some embodiments, the cannabinoid formulation comprises the cannabinoid is evenly dispersed within at least a portion of the unit dosage form. In some embodiments, the cannabinoid formulation comprises a signifier signifying a cannabinoid dosage is associated directly with the unit dosage form by embossing, or by colour, pattern, or shape feature. In some embodiments, the signifier is adapted to be directly interpreted by a consumer and/or is a machine-readable code. In some embodiments, the unit dosage form is contained in an individual blister pack sealed in an inert gas atmosphere comprising little or no oxygen. In some embodiments, the lipid carrier is present in the form of a nanoemulsion comprising lipid particles having an average particle size of about 20-100, 20-200, 20-300, 20-400, 20-500, 20-600, 100-300, 200-500, or 100-600 nm. In some embodiments, the lipid carrier comprises MCFAs comprising or consisting of 12-16 carbon atoms or LCFAs comprising or consisting of 13 or more carbon atoms. In some embodiments, the cannabinoid formulation comprises an enteric coating for containing the one or more cannabinoids and the lipid carrier, wherein the enteric coating degrades, dissolves, or otherwise provides a release of the cannabinoid and the lipid carrier in an environment having a pH value of below about 5.5, at about 5.5 or greater, at about 6.0 to about 6.5, at about 6.5 to 7.5, or greater than about 7.5.

In one aspect, described herein is a method of administering one or more cannabinoids to a subject, comprising orally administering to the subject a formulation comprising one or more cannabinoids selected from the group consisting of: 0.1-100 mg tetrahydrocannabinol (THC); 0.1-750 mg tetrahydrocannabinolic acid (THCA); 0.1-750 mg cannabidiol (CBD); 0.1-750 mg cannabidiolic acid (CBDA); and 0.1-750 mg cannabigerol (CBG); 1-750 mg cannabinchromene (CBC); and a lipid carrier comprising one or more lipids selected from the group consisting of: camelina oil; a marine phospholipid; fish oil; krill oil; flaxseed oil; an oil comprising an omeg-3 to omega-6 ratio of about 1.0 or higher, about 1.5 or higher, about 2.0 or higher, or about 2.2. or higher; a hydrolyzed oil; a vegetable oil; hemp oil; an oil containing medium-chain fatty acids (MCFAs); and an oil containing long-chain fatty acids (LCFAs). In some embodiments, the lipid carrier comprises or consists of camelina oil. Further described herein is a method of administering a therapeutically and/or psychotropically effective amount of one or more cannabinoids to a subject, comprising orally administering to the subject a cannabinoid formulation as described herein.

In some embodiments, the subject has a disease or disorder treatable by the administration of the one or more cannabinoids. In some embodiments, the disease or disorder is selected from the group consisting of: pain, inflammation, anxiety, depression, insomnia, sleep disorders, lack of energy, lack of alertness, weight gain, obesity, diabetes, metabolic syndrome, nausea (acute or anticipatory), epilepsy, spasticity, schizophrenia, bi-polar disorder, cancer and neoplasia, chronic pain, osteoarthritic pain, bacterial and/or fungal infection, fibromyalgia, appetite enhancement, appetite suppression, and any combination thereof. In some embodiments, the disease or disorder is selected from the group consisting of: pain, inflammation, anxiety, depression, insomnia, sleep disorders, lack of energy, lack of alertness, weight gain, obesity, diabetes, metabolic syndrome, nausea (acute or anticipatory), epilepsy, spasticity, schizophrenia, bi-polar disorder, cancer and neoplasia, chronic pain, osteoarthritic pain, fibromyalgia, appetite enhancement, appetite suppression, and any combination thereof.

In some embodiments, the formulation for oral administration comprises a unit dose or combination dose of cannabinoid(s) selected from the list consisting of (each cannabinoid milligram amount about or equal to):
25 mg THCA and 2 mg THC;
25 mg THC;
1 mg THCA, 25 mg CBDA, and 2 mg CBD;
1 m
g THCA and 25 mg CBD;
25 mg THCA, 2 mg THC, 25 mg CBDA, and 2 mg CBD;
25 mg THCA, 2 mg THC, and 2 mg CBD;
25 mg THC and 25 mg CBD;
25 mg THC and 2 mg CBD;
1 mg THC, 25 mg CBD; 25 mg CBG, and 25 mg CBC;
25 mg THC and 25 mg THCV;
9 mg THCA and 1 mg THC;
10 mg THC;
9 mg THCA, 1 mg THC, 9 mg CBDA, and 1 mg CBD;
9 mg THCA, 1 mg THC, and 10 mg CBD;
10 mg THC and 1 mg CBD;
10 mg THC and 10 mg THCV;
600 mg THC;
600 mg THCA and 50 mg THC;
100 mg THC;
600 mg CBDA;
25 mg THCA, 600 mg CBDA, and 60 mg CBD;
100 mg CBD;
4 mg THC and 100 mg CBD;
600 mg CBD;
25 mg THC and 600 mg CBD;
600 mg CBG;
300 mg THCA and 300 mg CBDA;
300 mg THCA, 30 mg THC, 300 mg CBDA, and 30 mg CBD;
300 mg THCA and 30 mg CBD;
300 mg THCA, 30 mg THC, and 300 mg CBD;
100 mg THC and 100 mg CBD;
100 mg THC and 30 mg CBD;
300 mg THC and 300 mg CBG;
300 mg THC and 300 mg CBC;
300 mg CBD and 300 mg CBG;
300 mg CBD and 300 mg CBC;
300 mg CBD, 300 mg CBG, and 300 mg CBC;
10 mg THC, 250 mg CBD, 250 mg CBG, and 250 mg CBC;
100 mg THC and 500 mg THCV;
300 mg CBD and 300 mg THCV; and
100 mg CBD and 100 mg THCV.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1: Plasma concentration-time profiles of Δ⁹-tetrahydrocannabinol (THC) following IV bolus (4 mg/kg, n=5), oral lipid-free formulation (12 mg/kg, n=6, and oral long-chain triglyceride (LCT)-based formulation (12 mg/kg, n=5) to rats. The data are shown as mean+/−S.E.M. Data taken from Zgair et al. Am J Transl Res 2016; 8(8):3448-3459.

FIG. 2: Plasma concentration-time profile of THC following oral gavage of THC (12 mg/kg) formulated with four different carrier oils (sesame, krill, camelina and emulsified camelina oil). Data is shown as mean+/−S.E.M.

FIG. 3: Plasma concentration-time profile of 11-OH-THC following oral gavage of THC (12 mg/kg) formulated with four different carrier oils (sesame, krill, camelina and emulsified camelina oil). Data is shown as mean+/−S.E.M.

FIG. 4: Plasma concentration-time profile of THC—COOH following oral gavage of THC (12 mg/kg) formulated with four different carrier oils (sesame, krill, camelina and emulsified camelina oil). Data is shown as mean+/−S.E.M.

### DETAILED DESCRIPTION OF THE INVENTION

The potential to use cannabinoids and carrier oils in combination has yet to be fully explored. The invention herein is directed to novel combinations comprising A) one or more carrier oils, and B) one or more cannabinoids, in oral formulation. Such products are useful as health products, dietary supplements, and for treatment of human diseases and disorders.

The invention herein provides novel oral formulations comprising a combination of one or more cannabinoids with one or more lipid carriers. The invention has a variety of advantages, including a surprising increase in cannabinoid bioavailability for the treatment of human diseases and disorders. In particular, the oral formulation combinations provided herein demonstrate increased bioavailability of cannabinoids for the treatment of pain, inflammation, anxiety, depression, insomnia, sleep disorders, lack of energy, lack of alertness, weight gain, obesity, diabetes and metabolic syndrome, acute and anticipatory nausea, epilepsy, spasticity, schizophrenia, bi-polar disorder, cancer and neoplasia, chronic pain, osteoarthritic pain, bacterial and/or fungal infection, fibromyalgia, appetite suppression, and/or to act as an anti-proliferative agent.

### Definitions

“Bioavailability” refers to the fraction or dose of an administered substance capable of being absorbed by the body's circulatory system and available for use or storage by a body (e.g., a cannabinoid). Bioavailability may be measured by calculating the concentration of a substance administered to a body over time. Cmax is a term used in pharmacokinetics refers to the maximum concentration that a compound/drug achieves in tested area after the drug has been administrated. The Cmax is often measured in an effort to show bioequivalence between a generic and innovator drug product. AUC (area under the curve) represents the total drug/compound exposure across time. AUC is proportional to the total amount of a compound absorbed by the body for a dose of a compound

“Cannabinoid” refers to a chemical compound that is capable of acting on cannabinoid receptors, for example in the brain. Cannabinoids may be produced naturally in the body (endocannabinoids), found in cannabis and some other plants (phytocannabinoids), or manufactured artificially (synthetic cannabinoids). Cannabinoids produced by cannabis during its cultivation and growth include tetrahydrocannabinolic acid (THCA), cannabinolic acid (CBNA), cannabidiolic acid (CBDA), cannabichromenic acid (CBCA), and cannabigerolic acid (CBGA). As used herein, cannabinoid may also refer to the corresponding decarboxylated moieties, tetrahydrocannabinol (THC), cannabinol (CBN), cannabidiol (CBD), cannabinchromene (CBC) and cannabigerol (CBG), (each of which may be derived from its parent compound by mild heating typically above 105° C.), and the corresponding liver metabolites that result upon oral consumption by humans of these compounds, such as but not limited to 11-OH-THC and THC—COOH (also known as 11-Nor-9-carboxyTHC). Cannabinoids may be synthesized by chemical or biological methods. Phytocannabinoids may be distinguished from endocannabinoids which are chemically distinct, are synthesized in mammalian cells from lipids and other macromolecule precursors, which are not phytocannabinoids, and which may be endogenous ligands of the CB1 and/or CB2 receptors.

“Cannabis” as used herein includes all members of the Cannabis genus, including without limitation Cannabis sativa, Cannabis indica, Cannabis ruderalis, and hybrids thereof. “Cannabis” also includes Charlotte's Web and other high CBD, low THCA plant varieties.

“Defined dose” means a dose of one or more active ingredients (typically cannabinoids) which has been selected during the production process and may be signified to a consumer by a signifier associated with the object.

“Effective dose” may refer to the amount of a cannabinoid substance sufficient to induce a change in an individual user. An effective dose may also mean an amount of a cannabinoid substance that is needed to provide a desired level of said cannabinoid in the bloodstream of an individual user to provide a desired response. An effective dose can be administered in one administration, or through multiple administrations of an amount that total an effective dose. The effective dose may be the result of empirical and/or individualized (case-by-case) determination on the part of an individual user.

“Enteric coating” refers to a barrier applied to an orally delivered substance that prevents or delays its dissolution or disintegration in the gastric environment. Enteric coatings can protect an orally delivered substance from the acidity of the stomach, protect the stomach from detrimental effects of an orally delivered substance, or delay the release of an orally delivered substance until after the stomach (ex. intestinal tract). Examples of enteric-coated dosage forms include without limitation tablets, mini-tablets, pellets, granules, capsules, gel capsules.

“Emulsion” refers to a mixture of two or more immiscible liquids in which one is present as droplets distributed throughout the other. The average droplet size in an emulsion may be between 100-1000 nm.

“Fatty acid” refers to a carboxylic acid with an aliphatic chain, which may be either saturated or unsaturated. The aliphatic chain may be short, medium, or long depending on the number of carbon atoms. For example, short chain fatty acids may comprise five or fewer carbon atoms, medium chain fatty acids may comprise 6-12 carbon atoms, and long chain fatty acids may comprise 13 or more carbon atoms.

“Lipid carrier” refers to lipid-based drug delivery systems including without limitation emulsions, nanoemulsions, lipid nanoparticles, solid lipid nanoparticles, nanostructured lipid carriers. Lipid carriers can be used to increase solubilization and absorption of hydrophobic compounds, which may result in enhanced bioavailability. Suitable lipids for use in formulating lipid carriers of the invention include, without limitation, camelina oil, marine phospholipids.

“Liposome” means a vesicle, which may be spherical, having at least one lipid bilayer. Liposomes can be used as a vehicle for delivery of nutrients and/or pharmaceutical agents.

“Micelle” means an aggregate (or supramolecular assembly) of surfactant molecules dispersed in a liquid colloid.

“Nanoemulsion” refers to a mixture of two or more immiscible liquids in which one is present as nano-sized microscopic droplets distributed throughout the other. The average droplet size in a nanoemulsion is usually between 20-600 nm.

“Oral Formulation” means a formulation which is conveniently administered orally to a human subject.

“Pharmacodynamic” parameters (PD) means dose-response relationships, that is, the relationships between a substances' plasma concentration and its effect.

“Pharmacokinetic” (PK) parameters are usually used to describe the rate of absorption of a substance into a biological system. Graphing a substance's serum concentration versus time reveals the drug's basic PK properties: the maximum concentration the drug attains (Cₘₐₓ), the time at which maximum concentration occurs (tₘₐₓ), and the area under the concentration-versus-time curve (AUC) which estimates total systemic exposure.

“Psychotropic dose” means a dose of a substance capable of affecting a user's mental state. Examples of psychotropic cannabinoids include without limitation THC, cannabinol (CBN).

“Unit dosage form” or “UDF” means a physically fixed unit dose of a formulation which is conveniently consumed by a consumer in unit form (e.g. requires no measuring or adjusting of dosage before consumption). A consumer may consume one or more UDFs at a time.

### Ingredients of the Unit Dosage Form

The unit dosage forms of the invention may comprise a lipid carrier, which may be formulated from one or more lipids including, without limitation, camelina oil and/or a marine phospholipid.

Camelina oil is an edible oil derived from the seeds of Camelina sativa. Camelina oil has been found to contain high amounts of omega-3 fatty acids and vitamin E, making it suitable for use as a nutritional supplement and as a general purpose oil. Due to its high vitamin E content, Camelina oil may be used as an antioxidant and as an anti-inflammatory. Camelina oil may also be prepared as an extract of a cultivated Camelina sativa plant crop.

Phospholipid extracts derived from natural marine or aquatic sources have been shown to possess a variety of nutraceutical and/or therapeutic properties. These marine extracts may comprise a variety of phospholipids, fatty acid, metals, and flavonoid compounds. Examples of suitable marine phospholipids for use in formulating the lipid carriers of the present invention are further described in U.S. Pat. Nos. 8,680,080 B2, 8,383,675 B2, and 8,278,351 B2 and others in the name of Neptune Tech and Bioresources Inc., all of which are incorporated herein by reference in their entirety.

The unit dosage forms of the invention may further comprise a defined dose of one or more cannabinoids selected from among the group consisting of:
0.1-750 mg tetrahydrocannabinolic acid (THCA),
0.1-100 mg tetrahydrocannabinol (THC),
0.1-750 mg cannabidiolic acid (CBDA),
0.1-750 mg cannabidiol (CBD); and
0.1-750 mg cannabigerol (CBG),

The cannabinoid(s) may be prepared as an extract of a cultivated cannabis plant crop (as described further below), or they may be synthetically prepared in a chemical process (as for example in patent applications WO2014134281, WO2015068052, WO2016030828 and others in the name of Full Spectrum Laboratories Limited (Dublin IE)), all of which are incorporated by reference in their entireties. When prepared as an extract, the composition may also comprise terpenes and other organic molecules co-extracted in the process.

As will be described further below, the unit dosage forms may also comprise diverse additional features which may include an anti-oxidant, other pharmaceutically acceptable additives, a carrier oil, an outer shell that is substantially opaque to one or both of ultraviolet and visible light, an enteric-coating, and/or a signifier which signifies the cannabinoid dosage of the unit dosage form, such as a signifier generated by embossing, or by colour, pattern or shape feature, which signifier may be adapted to be directly interpreted by a consumer and/or is a machine-readable code.

### Preferred Combinations

In some embodiments, the formulation consists of one or more cannabinoids, which in preferred embodiments comprise one or more members of the group consisting of THCA, THC, CBDA, CBD, CBC, and CBG, combined with a specific carrier oil, a particular lipid particle size, and a specific coating.

In some embodiments the product consists of one or more cannabinoids, which in preferred embodiments comprise one or more members of the group consisting of THCA, THC, CBDA, CBD, CBC, and CBG with camelina oil in a nanoemulsion with an average particle size of between 20 to 100 nm in a capsule or tablet with an enteric coating with a controlled release at pH values above 5.

In some embodiments the product consists of one or more cannabinoids, which in preferred embodiments comprise one or more members of the group consisting of THCA, THC, CBDA, CBD, CBC, and CBG with fish oil in a nanoemulsion with an average particle size of between 20 to 100 nm in a capsule or tablet with an enteric coating with a controlled release at pH values above 5.

In some embodiments the product consists of one or more cannabinoids, which in preferred embodiments comprise one or more members of the group consisting of THCA, THC, CBDA, CBD, CBC, and CBG with krill oil in a nanoemulsion with an average particle size of between 20 to 100 nm in a capsule or tablet with an enteric coating with a controlled release at pH values above 5.

In some embodiments the product consists of one or more cannabinoids, which in preferred embodiments comprise one or more members of the group consisting of THCA, THC, CBDA, CBD, CBC, and CBG with a medium chain fatty acid in a nanoemulsion with an average particle size of between 20 to 100 nm in a capsule or tablet with an enteric coating with a controlled release at pH values above 5.

In some embodiments the product consists of one or more cannabinoids, which in preferred embodiments comprise one or more members of the group consisting of THCA, THC, CBDA, CBD, CBC, and CBG with long chain fatty acid in a nanoemulsion with an average particle size of between 20 to 100 nm in a capsule or tablet with an enteric coating with a controlled release at pH values above 5.

In some embodiments the product consists of one or more cannabinoids, which in preferred embodiments comprise one or more members of the group consisting of THCA, THC, CBDA, CBD, CBC, and CBG with camelina oil in a nanoemulsion with an average particle size of between 20 to 200 nm in a capsule or tablet with an enteric coating with a controlled release at pH values above 5.

In some embodiments the product consists of one or more cannabinoids, which in preferred embodiments comprise one or more members of the group consisting of THCA, THC, CBDA, CBD, CBC, and CBG with fish oil in a nanoemulsion with an average particle size of between 20 to 200 nm in a capsule or tablet with an enteric coating with a controlled release at pH values above 5.

In some embodiments the product consists of one or more cannabinoids, which in preferred embodiments comprise one or more members of the group consisting of THCA, THC, CBDA, CBD, CBC, and CBG with krill oil in a nanoemulsion with an average particle size of between 20 to 200 nm in a capsule or tablet with an enteric coating with a controlled release at pH values above 5.

In some embodiments the product consists of one or more cannabinoids, which in preferred embodiments comprise one or more members of the group consisting of THCA, THC, CBDA, CBD, CBC, and CBG with a medium chain fatty acid in a nanoemulsion with an average particle size of between 20 to 200 nm in a capsule or tablet with an enteric coating with a controlled release at pH values above 5.

In some embodiments the product consists of one or more cannabinoids, which in preferred embodiments comprise one or more members of the group consisting of THCA, THC, CBDA, CBD, CBC, and CBG with long chain fatty acid in a nanoemulsion with an average particle size of between 20 to 200 nm in a capsule or tablet with an enteric coating with a controlled release at pH values above 5.

In some embodiments the product consists of one or more cannabinoids, which in preferred embodiments comprise one or more members of the group consisting of THCA, THC, CBDA, CBD, CBC, and CBG with camelina oil in a nanoemulsion with an average particle size of between 20 to 300 nm in a capsule or tablet with an enteric coating with a controlled release at pH values above 5.

In some embodiments the product consists of one or more cannabinoids, which in preferred embodiments comprise one or more members of the group consisting of THCA, THC, CBDA, CBD, CBC, and CBG with fish oil in a nanoemulsion with an average particle size of between 20 to 300 nm in a capsule or tablet with an enteric coating with a controlled release at pH values above 5.

In some embodiments the product consists of one or more cannabinoids, which in preferred embodiments comprise one or more members of the group consisting of THCA, THC, CBDA, CBD, CBC, and CBG with krill oil in a nanoemulsion with an average particle size of between 20 to 300 nm in a capsule or tablet with an enteric coating with a controlled release at pH values above 5.

In some embodiments the product consists of one or more cannabinoids, which in preferred embodiments comprise one or more members of the group consisting of THCA, THC, CBDA, CBD, CBC, and CBG with a medium chain fatty acid in a nanoemulsion with an average particle size of between 20 to 300 nm in a capsule or tablet with an enteric coating with a controlled release at pH values above 5.

In some embodiments the product consists of one or more cannabinoids, which in preferred embodiments comprise one or more members of the group consisting of THCA, THC, CBDA, CBD, CBC, and CBG with long chain fatty acid in a nanoemulsion with an average particle size of between 20 to 400 nm in a capsule or tablet with an enteric coating with a controlled release at pH values above 5.

In some embodiments the product consists of one or more cannabinoids, which in preferred embodiments comprise one or more members of the group consisting of THCA, THC, CBDA, CBD, CBC, and CBG with camelina oil in a nanoemulsion with an average particle size of between 20 to 400 nm in a capsule or tablet with an enteric coating with a controlled release at pH values above 5.

In some embodiments the product consists of one or more cannabinoids, which in preferred embodiments comprise one or more members of the group consisting of THCA, THC, CBDA, CBD, CBC, and CBG with fish oil in a nanoemulsion with an average particle size of between 20 to 400 nm in a capsule or tablet with an enteric coating with a controlled release at pH values above 5.

In some embodiments the product consists of one or more cannabinoids, which in preferred embodiments comprise one or more members of the group consisting of THCA, THC, CBDA, CBD, CBC, and CBG with krill oil in a nanoemulsion with an average particle size of between 20 to 400 nm in a capsule or tablet with an enteric coating with a controlled release at pH values above 5.

In some embodiments the product consists of one or more cannabinoids, which in preferred embodiments comprise one or more members of the group consisting of THCA, THC, CBDA, CBD, CBC, and CBG with a medium chain fatty acid in a nanoemulsion with an average particle size of between 20 to 400 nm in a capsule or tablet with an enteric coating with a controlled release at pH values above 5.

In some embodiments the product consists of one or more cannabinoids, which in preferred embodiments comprise one or more members of the group consisting of THCA, THC, CBDA, CBD, CBC, and CBG with long chain fatty acid in a nanoemulsion with an average particle size of between 20 to 400 nm in a capsule or tablet with an enteric coating with a controlled release at pH values above 5.

In some embodiments the product consists of one or more cannabinoids, which in preferred embodiments comprise one or more members of the group consisting of THCA, THC, CBDA, CBD, CBC, and CBG with camelina oil in a nanoemulsion with an average particle size of between 20 to 500 nm in a capsule or tablet with an enteric coating with a controlled release at pH values above 5.

In some embodiments the product consists of one or more cannabinoids, which in preferred embodiments comprise one or more members of the group consisting of THCA, THC, CBDA, CBD, CBC, and CBG with fish oil in a nanoemulsion with an average particle size of between 20 to 500 nm in a capsule or tablet with an enteric coating with a controlled release at pH values above 5.

In some embodiments the product consists of one or more cannabinoids, which in preferred embodiments comprise one or more members of the group consisting of THCA, THC, CBDA, CBD, CBC, and CBG with krill oil in a nanoemulsion with an average particle size of between 20 to 500 nm in a capsule or tablet with an enteric coating with a controlled release at pH values above 5.

In some embodiments the product consists of one or more cannabinoids, which in preferred embodiments comprise one or more members of the group consisting of THCA, THC, CBDA, CBD, CBC, and CBG with a medium chain fatty acid in a nanoemulsion with an average particle size of between 20 to 500 nm in a capsule or tablet with an enteric coating with a controlled release at pH values above 5.

In some embodiments the product consists of one or more cannabinoids, which in preferred embodiments comprise one or more members of the group consisting of THCA, THC, CBDA, CBD, CBC, and CBG with long chain fatty acid in a nanoemulsion with an average particle size of between 20 to 500 nm in a capsule or tablet with an enteric coating with a controlled release at pH values above 5.

In some embodiments the product consists of one or more cannabinoids, which in preferred embodiments comprise one or more members of the group consisting of THCA, THC, CBDA, CBD, CBC, and CBG with camelina oil in a nanoemulsion with an average particle size of between 20 to 600 nm in a capsule or tablet with an enteric coating with a controlled release at pH values above 5.

In some embodiments the product consists of one or more cannabinoids, which in preferred embodiments comprise one or more members of the group consisting of THCA, THC, CBDA, CBD, CBC, and CBG with fish oil in a nanoemulsion with an average particle size of between 20 to 600 nm in a capsule or tablet with an enteric coating with a controlled release at pH values above 5.

In some embodiments the product consists of one or more cannabinoids, which in preferred embodiments comprise one or more members of the group consisting of THCA, THC, CBDA, CBD, CBC, and CBG with krill oil in a nanoemulsion with an average particle size of between 20 to 600 nm in a capsule or tablet with an enteric coating with a controlled release at pH values above 5.

In some embodiments the product consists of one or more cannabinoids, which in preferred embodiments comprise one or more members of the group consisting of THCA, THC, CBDA, CBD, CBC, and CBG with a medium chain fatty acid in a nanoemulsion with an average particle size of between 20 to 600 nm in a capsule or tablet with an enteric coating with a controlled release at pH values above 5.

In some embodiments the product consists of one or more cannabinoids, which in preferred embodiments comprise one or more members of the group consisting of THCA, THC, CBDA, CBD, CBC, and CBG with long chain fatty acid in a nanoemulsion with an average particle size of between 20 to 600 nm in a capsule or tablet with an enteric coating with a controlled release at pH values above 5.

### Therapeutic Use of Unit Dosage Forms of the Invention

The oral formulations of the present invention may be used for the treatment or amelioration in humans and other animals of a range of diseases and conditions. The inventors have identified the advantages of the proposed novel combinations based on a variety of technical assessments which demonstrate surprising effects of the oral combinations on cannabinoid bioavailability relevant to the treatment or amelioration of a range of diseases and conditions. These assessments, described below, include 1) Identifying improved bioavailability and enhanced PK/PD of the combinations in in vivo models; and 2) By providing animal (including human) testing in complex diseases and disorders and models of complex diseases and disorders.

### Therapeutic Effects of Cannabinoids

The therapeutic effects of cannabinoids are becoming increasingly well known.

Cannabinoids such as THC, CBD, CBG, and CBN interact with the body in different ways, leading to a variety of cannabinoid-associated effects. For example, ingestion of THC may lead to increased relaxation or stress reduction, enhanced feelings of well-being and euphoria. THC has been used to treat ailments such as neuropathic pain, multiple sclerosis, Crohn's disease, in cancer patients to increase appetite and reduce nausea, inflammation (swelling and redness), and muscle control problems. CBD has been shown to alleviate certain adverse effects of THC experienced by some consumers. CBD can act as a sedative, anti-convulsive, anti-inflammatory, anti-oxidant, or anti-depressant. CBG is an adrenergic, serotonergic, enzyme modulator, slow the proliferation of cancer cells, reduces intraocular eye pressure caused by glaucoma, inflammatory bowel disease, Crohn's disease and analgesic, while CBN may regulate the immune system and induce sleep. CBC is a non-psychoactive cannabinoid widely considered to interact with the endocannabinoid system (ECS) through stimulation of the body's naturally occurring endocannabinoids, anandamide and 2-AG, and is a known agonist to TRPV1 and TRPA1 receptors (A. A. Izzo et al.: Br. J. Pharmacol. 166, 1444 (2012)). Additionally, CBC is thought to be a selective CB2 receptor agonist which may have therapeutic implications for the treatment of pain and inflammatory conditions through CB2-mediated regulatory pathways (M. Udoh et al.: Br. J. Pharmacol. (2019). Furthermore, CBC co-administered with THC produced an enhanced anti-inflammatory effect, suggesting a potential pharmacokinetic interaction between the two molecules (G. T. DeLong et al.: Drug Alcohol Depend. 112, 126 (2010). CBC has been identified as a molecule of interest for various therapeutic applications including pain, inflammation, digestive and gastrointestinal disorders. Furthermore, it is known to have antibacterial and antifungal effects, and could potentially contribute to the regeneration of brain cells, which possibly has implications in the treatments of multiple sclerosis bacterial and/or fungal infection, fibromyalgia, dementia, Alzheimer's and other neurodegenerative related conditions.

Unit dosage forms of the present invention may be applicable to a wide range of diseases or conditions, including without limitation, inflammatory, immune, neurological, psychiatric disorders, seizures/epilepsy, anxiety and stress disorders, malignancies, metabolic disorders, nutritional deficiencies, infectious diseases, types of gastrointestinal disorders, sleep disorders, cardiovascular disorders, complex chronic diseases, and various types of pain, including chronic and neuropathic pain.

More particularly, unit dosage forms of the present invention may be applicable, although not only, to at least one of pain, inflammation, anxiety, depression, insomnia, sleep disorders, lack of energy, lack of alertness, weight gain, obesity, diabetes and metabolic syndrome, acute and anticipatory nausea, epilepsy, spasticity, schizophrenia, bi-polar disorder, cancer and neoplasia, chronic pain, osteoarthritic pain, bacterial and/or fungal infection, fibromyalgia, all the foregoing in acute and/or chronic presentation, and may be further used to induce appetite suppression and/or act as an anti-proliferative agent.

### Dose Selection

Compositions and methods of the invention feature a combination of lipid carrier(s) and one or more cannabinoids.

The combinations of the invention may comprise one or more cannabinoids selected from among the group consisting of:
0.1-750 mg tetrahydrocannabinolic acid (THCA),
0.1-100 mg tetrahydrocannabinol (THC),
0.1-750 mg cannabidiolic acid (CBDA),
0.1-750 mg cannabidiol (CBD);
0.1-750 mg cannabinchromene (CBC), and
0.1-750 mg cannabigerol (CBG).

In some embodiments, the oral combinations may comprise a defined dose selected from the following ranges (which may be referred to as “low dose”): about 0 mg, 1 mg, 2, 3, 4, 5, 6, 7, 8, 9, or 10 mg, 20 mg, 30 mg, 40 mg, 50 mg, 60 mg, 70 mg, 80 mg, 90 mg, 100 mg, or any about any 1 mg interval between 0 mg and 100 mg THC, about 0 mg, 1 mg, 2, 3, 4, 5, 6, 7, 8, 9, or 10 mg, 20 mg, 30 mg, 40 mg, 50 mg, 60 mg, 70 mg, 80 mg, 90 mg, 100 mg, or any about any 1 mg interval between 0 mg and 100 mg THCA, about 0 mg, about 7 mg, about 75 mg, about 1 mg, 2, 3, 4, 5, 6, 7, 8, 9, or 10 mg, 20 mg, 30 mg, 40 mg, 50 mg, 60 mg, 70 mg, 80 mg, 90 mg, 100 mg, or about any 1 mg interval between 0 mg and 100 mg CBD, and/or about 0 mg, about 7 mg, about 75 mg, about 1 mg, 2, 3, 4, 5, 6, 7, 8, 9, or 10 mg, 20 mg, 30 mg, 40 mg, 50 mg, 60 mg, 70 mg, 80 mg, 90 mg, 100 mg, or about any 1 mg interval between 0 mg and 100 mg CBDA, or 10 mg, 20 mg, 30 mg, 40 mg, 50 mg, 60 mg, 70 mg, 80 mg, 90 mg, 100 mg, or about any 1 mg interval between 0 mg and 100 mg CBG, and/or about 0 mg, about 7 mg, about 75 mg, about 1 mg, 2, 3, 4, 5, 6, 7, 8, 9, or 10 mg, 20 mg, 30 mg, 40 mg, 50 mg, 60 mg, 70 mg, 80 mg, 90 mg, 100 mg, or about any 1 mg interval between 0 mg and 100 mg CBC. In some embodiments, the oral combinations comprise about 0 mg, 1 mg, 2, 3, 4, 5, 6, 7, 8, 9, or 10 mg, 20 mg, 30 mg, 40 mg, 50 mg, 60 mg, 70 mg, 80 mg, 90 mg, 100 mg, or about any 1 mg interval between 0 mg and 100 mg of one of the foregoing compounds. In some embodiments, the oral combinations of the present invention have defined dosages for more than one of the foregoing compounds. For example, in some embodiments, the oral combinations comprise from about 0 mg-1 mg, or any 0.1 mg interval therebetween THC, about 0 mg, about 9 mg, about 90 mg, about 1 mg, 2, 3, 4, 5, 6, 7, 8, 9, or 10 mg, 20 mg, 30 mg, 40 mg, 50 mg, 60 mg, 70 mg, 80 mg, 90 mg, 100 mg, or any about any 1 mg interval between 0 mg and 100 mg, about 0 mg, about 7 mg, about 75 mg, about 1 mg, 2, 3, 4, 5, 6, 7, 8, 9, or 10 mg, 20 mg, 30 mg, 40 mg, 50 mg, 60 mg, 70 mg, 80 mg, 90 mg, 100 mg, or any about any 1 mg interval between 0 mg and 100 mg CBD, and/or about 0 mg, about 7 mg, about 75 mg, about 1 mg, 2, 3, 4, 5, 6, 7, 8, 9, or 10 mg, 20 mg, 30 mg, 40 mg, 50 mg, 60 mg, 70 mg, 80 mg, 90 mg, 100 mg, or any about any 1 mg interval between 0 mg and 100 mg CBDA. In some embodiments, the oral combinations comprise from about 0 mg, about 9 mg, about 90 mg, about 1 mg, 2, 3, 4, 5, 6, 7, 8, 9, or 10 mg, 20 mg, 30 mg, 40 mg, 50 mg, 60 mg, 70 mg, 80 mg, 90 mg, 100 mg, or any about any 1 mg interval between 0 mg and 100 mg THC, 0 mg-1 mg THCA, or any 0.1 mg interval therebetween, about 0 mg, about 7 mg, about 75 mg, about 1 mg, 2, 3, 4, 5, 6, 7, 8, 9, or 10 mg, 20 mg, 30 mg, 40 mg, 50 mg, 60 mg, 70 mg, 80 mg, 90 mg, 100 mg, or any about any 1 mg interval between 0 mg and 100 mg CBD, and about 0 mg, about 7 mg, about 75 mg, about 1 mg, 2, 3, 4, 5, 6, 7, 8, 9, or 10 mg, 20 mg, 30 mg, 40 mg, 50 mg, 60 mg, 70 mg, 80 mg, 90 mg, 100 mg, or any about any 1 mg interval between 0 mg and 100 mg CBDA. In some embodiments, the compositions are substantially free of THC-type cannabinoid compounds. For example, in some embodiments the oral combinations comprise from about 0 mg-1 mg, or any 0.1 mg interval therebetween THC, 0 mg-1 mg THCA, or any 0.1 mg interval therebetween, about 0 mg, about 7 mg, about 75 mg, about 1 mg, 2, 3, 4, 5, 6, 7, 8, 9, or 10 mg, 20 mg, 30 mg, 40 mg, 50 mg, 60 mg, 70 mg, 80 mg, 90 mg, 100 mg, or any about any 1 mg interval between Omg and 100 mg CBD, and about 0 mg, about 7 mg, about 75 mg, about 1 mg, 2, 3, 4, 5, 6, 7, 8, 9, or 10 mg, 20 mg, 30 mg, 40 mg, 50 mg, 60 mg, 70 mg, 80 mg, 90 mg, 100 mg, or any about any 1 mg interval between Omg and 100 mg CBDA.

In some embodiments, the oral combinations may comprise a defined dose selected from the following ranges (which may be referred to as “high dose”): about 10 mg, 15, 20, 25, 30, 40, 50, 60, 70, 80, 90, or 100 mg, 200 mg, 300 mg, 400 mg, 500 mg, 600 mg, 700 mg, 750 mg, or any about any 10 mg interval between 0 mg and 750 mg THCA, about 0 mg, 1 mg, 2, 3, 4, 5, 6, 7, 8, 9, or 10 mg, 20 mg, 30 mg, 40 mg, 50 mg, 60 mg, 70 mg, 80 mg, 90 mg, 100 mg, or any about any 1 mg interval between 0 mg and 100 mg THC, about 10 mg, 15, 20, 25, 30, 40, 50, 60, 70, 80, 90, or 100 mg, 200 mg, 300 mg, 400 mg, 500 mg, 600 mg, 700 mg, 750 mg, or any about any 10 mg interval between 0 mg and 750 mg CBD, and/or about 10 mg, 15, 20, 25, 30, 40, 50, 60, 70, 80, 90, or 100 mg, 200 mg, 300 mg, 400 mg, 500 mg, 600 mg, 700 mg, 750 mg, or any about any 10 mg interval between 0 mg and 750 mg CBDA, or about 10 mg, 15, 20, 25, 30, 40, 50, 60, 70, 80, 90, or 100 mg, 200 mg, 300 mg, 400 mg, 500 mg, 600 mg, 700 mg, 750 mg, or any about any 10 mg interval between 0 mg and 750 mg CBG, and/or about 10 mg, 15, 20, 25, 30, 40, 50, 60, 70, 80, 90, or 100 mg, 200 mg, 300 mg, 400 mg, 500 mg, 600 mg, 700 mg, 750 mg, or any about any 10 mg interval between 0 mg and 750 mg CBC. In some “high dose” embodiments, the oral combinations comprise about 10 mg, 15, 20, 25, 30, 40, 50, 60, 70, 80, 90, or 100 mg, 200 mg, 300 mg, 400 mg, 500 mg, 600 mg, 700 mg, 750 mg, or any about 10 mg interval between 0 mg and 750 mg of one of the foregoing compounds. In some “high dose” embodiments, the oral combinations of the present invention have defined dosages of more than one of the cannabinoids. In some high dose embodiments, the compositions are substantially free of THC-type cannabinoid compounds. For example, in some embodiments the oral combinations comprise from about 0 mg-1 mg, or any 0.1 mg interval therebetween THC, 0 mg-1 mg THCA, or any 0.1 mg interval therebetween, plus CBD in the amount of about 0 mg, about 25 mg, about 75 mg, about 10 mg, 20, 30, 40, 50, 60, 70, 80, 90, or 100 mg, 200 mg, 300 mg, 400 mg, 500 mg, 600 mg, 700 mg, 750 mg, or any about 10 mg interval between 0 mg and 750 mg, and/or CBDA in the amount of about 0 mg, about 25 mg, about 75 mg, about 10 mg, 20, 30, 40, 50, 60, 70, 80, 90, or 100 mg, 200 mg, 300 mg, 400 mg, 500 mg, 600 mg, 700 mg, 750 mg.

In some embodiments, the oral combinations described herein comprise an “effective” amount of one or more of the cannabinoid ingredients described herein. The term “effective amount” refers to an amount of the one or more cannabinoid ingredients sufficient to induce a response in an individual user, either subjectively or objectively determined. An effective amount also means an amount of the one or more cannabinoid ingredients that is needed to provide a desired level of cannabinoid(s) in the bloodstream of an individual user to provide an anticipated physiological response. An effective amount of a cannabinoid ingredient can be administered in one administration, or through multiple administrations of an amount that totals an effective amount, preferably within a 24-hour period. It is understood that the effective amount can be the result of empirical and/or individualized (case-by-case) determination on the part of the individual user. For example, a therapeutically effective amount of said one or more cannabinoid ingredients may be in the range of about 1 mg to 2,000 mg, or any 1 mg or 10 mg interval there between total cannabinoids per day.

In some low dose embodiments, an effective amount of one or more cannabinoid ingredients may be in the range of about 1 mg-5 mg, or any 1 mg or 0.1 mg interval therebetween per day. For example, for an adult, about 1-2 mg, or 0.1 mg interval therebetween, a day total cannabinoids may provide a very low-end dose below the psychoactive threshold.

In some embodiments, an effective amount of THC may be in the range of about 5 mg-25 mg, or any 1 mg interval therebetween. For example, most vapers inhale about 10 to 30 mg of THC to establish a mild, temporary, psychoactive effect. In a high dose embodiment the oral formulation may contain THC in an amount of 25 mg to 100 mg.

In some embodiments, a composition of the present invention may comprise THCA in an amount between 5-200 mg, THC in an amount less than 1.0 mg, and CBDA in an amount between 0.1-600 mg, and have a total mass of 100-750 mg.

In some embodiments, a composition of the present invention may comprise THCA in an amount less than 5.0 mg, THC in an amount between 5-30 mg, and CBD in an amount between 0.1-600 mg, and have a total mass of 100-750 mg.

In some embodiments, a composition of the present invention may comprise THCA in an amount less than 1.0 mg, THC in an amount less than 1.0 mg, and CBD in an amount between 5-600 mg, and have a total mass of 100-750 mg.

In some embodiments, a composition of the present invention may comprise THCA in an amount less than 1.0 mg, THC in an amount less than 1.0 mg, and CBG in an amount between 5-600 mg, and have a total mass of 100-750 mg.

In some embodiments, an effective amount of CBD for treating conditions or disorders disclosed elsewhere herein may be in the low dose range of about 0 mg, about 7 mg, about 75 mg, about 1 mg, 2, 3, 4, 5, 6, 7, 8, 9, or 10 mg, 20 mg, 30 mg, 40 mg, 50 mg, 60 mg, 70 mg, 80 mg, 90 mg, 100 mg, or any about any 1 mg interval between 0 mg and 100 mg per day. Preferably, the low dose amount of CBD may be about 50 mg per day. For example, a recommended CBD dosage standard may be about 25 mg of CBD taken twice a day.

Alternatively, in some embodiments, an effective amount of CBD for treating conditions or disorders disclosed elsewhere herein may be in the high dose range of about 50-2000 mg/day or higher. Such effective amounts may be provided by ingestion of multiple oral dosage forms comprising CBD in the amount of, about 50 mg, about 75 mg, about 100 mg, 200 mg, 250 mg, 300 mg, 400 mg, 500 mg, 600 mg, 700 mg, 750 mg, or any about any 10 mg interval between 100 mg and 750 mg.

In some embodiments, an effective amount of THCA may be in the range of about 0 mg, about 9 mg, about 90 mg, about 1 mg, 2, 3, 4, 5, 6, 7, 8, 9, or 10 mg, 20 mg, 30 mg, 40 mg, 50 mg, 60 mg, 70 mg, 80 mg, 90 mg, 100 mg, or any about any 1 mg interval between 0 mg and 100 mg. In some embodiments, only one cannabinoid will be present at a physiologically relevant level, in other embodiments two or more cannabinoids may be present at physiologically relevant levels. The second cannabinoid may be one previously cited, or may be an alternative cannabinoid which may also be isolated from or extracted from cannabis, or may be a simple chemical derivative thereof.

Tables 1A-1C provide examples for preferred embodiments.

TABLE 1A

| | Predominant | | | | | | | | |
|---|---|---|---|---|---|---|---|---|---|
| Form | Cannabinoid | Active Ingredient (mg/cap) | | | | | | | |
| # | (s) | THCA | THC | CBDA | CBD | CBG | CBC | THCV | Indication |
| 1 | THCA | 25 | 2 | | | | | | Pain, Acute and anticipatory nausea |
| 2 | THC | | 25 | | | | | | Pain, Appetite enhancement |
| 3 | CBDA | 1 | | 25 | 2 | | | | Acute and anticipatory nausea |
| 4 | CBD | | 1 | | 25 | | | | Anxiety, Sleep |
| 5 | THCA:CBDA | 25 | 2 | 25 | 2 | | | | Acute and anticipatory nausea |
| 6 | THCA:CBD | 25 | 2 | | 25 | | | | Pain; Anxiety; Sleep |
| 7 | THC:CBD | | 25 | | 25 | | | | Pain; Anxiety; Sleep |
| 8 | THC:CBD | | 25 | | 2 | | | | Energy |
| 9 | CBD:CBG:CBC | | 1 | | 25 | 25 | 25 | | Osteoarthritic Pain |
| 10 | THC:THCV | | 25 | | | | | 25 | Energy |

TABLE 1B
250 mg capsule (low dose; 10 mg THC maximum)

| | Predominant | | | | | | | | |
|---|---|---|---|---|---|---|---|---|---|
| Form | Cannabinoid | | | | | | | | |
| # | (s) | THCA | THC | CBDA | CBD | CBG | CBC | THCV | Indication |
| 11 | THCA | 9 | 1 | | | | | | Pain, Acute and anticipatory nausea |
| 12 | THC | | 10 | | | | | | Pain, Appetite enhancement |
| 13 | THCA:CBDA | 9 | 1 | 9 | 1 | | | | Acute and anticipatory nausea |
| 14 | THCA:CBD | 9 | 1 | | 10 | | | | Pain; Anxiety; Sleep |
| 15 | THC:CBD | | 10 | | 10 | | | | Pain; Anxiety; Sleep |
| 16 | THC:CBD | | 10 | | 1 | | | | Energy |
| 17 | THC:THCV | | 10 | | | | | 10 | Energy |

TABLE 1C
1000 mg capsule (high dose)

| | Predominant | | | | | | | | |
|---|---|---|---|---|---|---|---|---|---|
| Form | cannabinoid | | | | | | | | |
| # | (s) | THCA | THC | CBDA | CBD | CBG | CBC | THCV | Indication/function |
| 19 | THCA | 600 | | | | | | | Pain; Acute and anticipatory |
| | | | | | | | | | nausea |
| 20 | THCA:THC | 600 | 60 | | | | | | Pain |
| 21 | THC | | 100 | | | | | | Pain |
| 22 | CBDA | | | 600 | | | | | Acute and anticipatory nausea |
| 23 | CBDA:CBD | 25 | | 600 | 60 | | | | Acute and anticipatory nausea |
| 24 | CBD | | | | 100 | | | | Anti-epileptic |
| 25 | CBD | | 4 | | 100 | | | | Anti-epileptic |
| 26 | CBD | | | | 600 | | | | Anti-epileptic |
| 27 | CBD | | 25 | | 600 | | | | Chronic Pain; Inflammation; |
| | | | | | | | | | Schizophrenia; Cancer |
| | | | | | | | | | Anti-proliferative |
| 28 | CBG | | | | | 600 | | | Cancer Anti-proliferative; |
| | | | | | | | | | Antimicrobial; bone stimulant |
| 29 | THCA:CBDA | 300 | | 300 | | | | | Acute and anticipatory nausea |
| 30 | THCA:CBDA | 300 | 30 | 300 | 30 | | | | Acute and anticipatory nausea |
| 31 | THCA:CBD | 300 | | | 300 | | | | Pain |
| 32 | THCA:CBD | 300 | 30 | | 300 | | | | Pain |
| 33 | THC:CBD | | 100 | | 100 | | | | Pain; Spasticity |
| 34 | THC:CBD | | 100 | | 30 | | | | Pain |
| 35 | THC:CBG | | 300 | | | 300 | | | Pain; Cancer Anti-proliferative |
| 36 | THC:CBC | | 300 | | | | 300 | | Pain; Anti-Inflammatory |
| 37 | CBD:CBG | | | | 300 | 300 | | | Pain; Cancer Anti-proliferative |
| 38 | CBD:CBC | | | | 300 | | 300 | | Pain; Anti-Inflammatory |
| 39 | CBD:CBG:CBC | | | | 300 | 300 | 300 | | Osteoarthritic Pain; |
| | | | | | | | | | Anti-proliferative, sleep |
| 40 | CBD:CBG:CBC | | 10 | | 250 | 250 | 250 | | Osteoarthritic Pain; |
| | | | | | | | | | Anti-proliferative, sleep |
| 41 | THC:THCV | | 100 | | | | | 500 | Pain |
| 42 | CBD:THCV | | | | 300 | | | 300 | Appetite suppression; |
| 43 | CBD:THCV | | | | 100 | | | 100 | Anti-epileptic |

The precise amount of cannabinoid required for an effective dose in an individual will depend upon numerous factors, e.g. type of cannabinoid(s) and type or combination of lipid carrier(s), type of lipid carrier, and the effect of the lipid carrier-cannabinoid combination on bioavailability. This disclosure provides UDFs suitable to obtain an effective dose which can be determined subjectively by the user or objectively by methods known to those skilled in the art. An achievement of the invention is that by using the UDFs of the invention, users and medical advisors for the first time have knowledge of and certainty with the preferred doses of cannabinoid they are employing with the lipid carrier. This is preferably achieved with a signifier identifying dosage of one or more components, as detailed further below.

### Source and Quality of Cannabinoid

The cannabinoid(s) for use in the present UDFs may be prepared by a variety of methods. It may be provided in the original plant form, preferably dried and cured into a flowable powder suitable for encapsulating. An alternative preferred method is by extraction from a cultivated cannabis crop. Organic extraction is a preferred method, although aqueous extraction, typically employed to prepare hashish, is also possible. Organic extraction can be performed with a wide variety of organic solvents or super-critical carbon dioxide, and at a variety of temperatures and under a variety of conditions. (Fairbairn and Liebmann (1973) J. Pharm. Pharmac. 25:150-155; Romano and Hazekamp (2013) Cannabinoids. 1(1)-1-11; Rovetto and Aieta (2017). J. Supercritical Fluids. 129: 16-27.), all of which are incorporated herein in their entireties. The resulting organic solvent-based extract can be, at room temperature, a liquid oil, or solid form wax, budder, or shatter depending on the conditions employed (which significantly impact the other plant alkyloids and polymers extracted by the process). Historically, less than 50% of cannabinoids were extracted from dried plant material. Fairbairn and Liebmann (1973), incorporated herein in its entirety. Modern techniques may extract over 90%.

The unpredictability of cultivated cannabis provides another potential challenge to formulation of the disclosed UDFs. As is well known, the most common varieties C. sativa, C. indica and C. ruderalis, have distinct (but overlapping) ranges of cannabinoids. Varieties and strains which are crossed or hybridized generate further different cannabinoid ratios. And, the cannabinoid ratios and overall amounts within a single variety are strongly influenced by the conditions of cultivation, especially light cycle, temperature, soil condition, nutrient availability, timing of harvest and pathogen exposure. The result is that a cultivated cannabis plant can have, by dry weight, anywhere from 0% up to greater than 30% of selected cannabinoids, and the ratios between individual cannabinoids can be highly diverse.

Preferred cannabis sp. cultivars for use preparing cannabinoid extracts include: Acapulco Gold; Afghani; African; Cambodian red; Columbian; Hawaiian; Jamaican gold; Mexican red; Panama red; Thai stick; Amnesia; AK-47; Amnesia Haze; Blueberry; Blue Dream; Bubba Kush; Bubblegum; Critical Mass; Durban Poison; Gorilla Glue; Haze; Hindu Kush; Jack Herer; Maui Waui; Northern Lights; OG Kush; Purple Haze; and Skunk. Preferred for cultivation in Canada are: Altair, Angie, CS, Carmagnola, Carmen, Deni, ESTA-1, FINOLA, Fasamo, Fedrina 74, Felina 34, Fibranova, Fibriko, Fibrimon 24, Fibrimon 56, Georgina, GranMa, Grandi, Judy, Katani, Kompolti, Kompolti Hibrid TC, Kompolti Sargaszaru, Laura Secord, Lovrin 110, Martha, Petera, Picolo, Quida, UC-RGM, Uniko B, Victoria, and Yvonne.

Preferred cannabis sp. cultivars for use in preparing CBD extracts that contain little or no THC or THCA include: Charlotte's Web and cultivars approved in Canada including CFX-1, CFX-2, CRS-1, Canda, Crag, Joey, USO 14, USO 31, X-59 (Hemp Nut), Delores, Silesia, Alyssa, Zolotonosha 11, Anka, Jutta, CanMa, and Ferimon.

Another critical aspect of the cannabinoid preparation is that during the cultivation phase, cannabis naturally synthesizes tetrahydrocannabinolic acid (THCA) and cannabidiolic acid (CBDA). These compounds convert respectively to THC (the primary psychoactive cannabinoid), and CBD (the non-psychoactive analgesic and anti-inflammatory cannabinoid) via decarboxylation. Decarboxylation may be induced by heating over 105° C. and/or by exposure to ultraviolet (UV) light. Importantly, gastric acids do not decarboxylate THCA or CBDA. (See Wang et al. (2016) Decarboxylation Study of Acidic Cannabinoids: A Novel Approach Using Ultra-High-Performance Supercritical Fluid Chromatography/Photodiode Array-Mass Spectrometry. Cannabis Cannabinoid Res.; 1(1): 262-271.) Therefore, a critical aspect of extracted cannabinoids is quality control on whether the harvested crop has been subjected to decarboxylating conditions that would decarboxylate THCA and CBDA to THC and CBD, respectively.

Because of the unpredictability of cannabis cultivation, preferably, extract preparations of cannabinoid(s) for use in UDFs of the present disclosure are analyzed to determine the precise concentrations of relevant cannabinoids, especially THCA, THC, CBDA and CBD, for use in preparing unit dosage forms of the invention.

As described above, the cannabinoids in the oral formulation of the present invention may be provided as dried plant matter, as extracts of plant matter, or as generated by chemical synthesis. A valuable parameter for usefulness is on whether the format is flowable. The cannabinoid format is preferably efficient for mixing with other components and for loading into capsules, forming tablets, and the like. Stickiness of powders or high-viscosity of liquids can be a deterrent to efficient preparation. The invention therefore takes advantage cannabinoid formats that are sufficiently flowable for use in manufacturing the UDFs described herein. Flowability of dried plant material may be enhanced by appropriate grinding and by addition of excipients, including but not limited to those described herein. Flowability of oil extracts may be enhanced by diluents, gliders and the like. Oil extracts with hydrophobic components may be mixed with powders to provide a dry flowable powder which can easily be mixed with other UDF components. (e.g., US Pat App. Publications 20170232210 and 20160243177). Alternatively, oil extracts may be spray dried with flowable particles to create a flowable powder format. In one preferred embodiment, extracts may be used to produce crystallized pure cannabinoids. Crystallized CBD can be prepared by high-vacuum treatment of extracts, as exemplified at https://www.leafscience.com/2017/11/06/cbd-isolate-powder/(viewed 22 Aug. 2018). Crystallized forms can be rendered into a suitable flowable powder by techniques common in the capsule/tablet industry.

### Carrier Oils

It should be appreciated that the oral formulations of the present invention may comprise at least one carrier oil. This feature is particularly advantageous as the carrier oil(s) may increase the bioavailability of one or more cannabinoid ingredients.

A wide variety of carrier oils may be employed to dissolve, solubilize or otherwise formulate the components of UDFs of the present disclosure into a liquid or semi-solid formulation suitable for manufacturing the oral formulation and unit dosage forms of the invention. Carrier oils may comprise short chain, medium chain and/or long chain fatty acids. Typically, carrier oils comprise, by mass, from about 50% to about 98%, about 75% to about 95%, and preferably about 90% to about 93% of the liquid fill of the UDF. The oils may be formulated with the cannabinoid(s) and the other components of the invention through any known formulation process, including but not limited to surfactant mixtures, emulsions, liposomes (e.g. fully encapsulated or lipid-aggregated), and nanoparticles.

Carrier oil(s) for use in the presently disclosed formulations may be plant-based or animal-based. Plant-based carrier oils can be derived from the fatty portions of a plant, for example, from the seeds, kernels or nuts. Animal-based carrier oils can be derived from raw animal fat or from the fatty tissues of an animal, for example, the liver.

A carrier oil of the present invention may exist in a variety of forms. For example, a carrier oil may be an oil, fat, wax, or similar product, for example, lecithin. A carrier oil may be hydrolyzed, hydrogenated, or non-hydrogenated. A hydrolyzed carrier oil refers to a carrier oil that has been split into its component parts in the presence of an aqueous base. Examples of component parts of a carrier oil may include glycerol, fatty acids etc. A hydrogenated carrier oil refers to an oil with saturated fatty acids that may be solid at room temperature. A non-hydrogenated carrier oil refers to an oil with unsaturated fatty acids that may be a liquid at room temperature. A non-hydrogenated carrier oil with unsaturated fatty acids can be chemically changed through the addition of hydrogen to form a hydrogenated carrier oil with saturated fatty acids.

In some embodiments, the carrier may be a bioactive or nutraceutical agent. Nutraceutical agents are natural, bioactive chemical compounds that have health promoting, disease preventing or medicinal properties

In some embodiments, oral formulations may comprise one carrier oil. In some embodiments, oral formulations may comprise a mixture of at least two carrier oils. A mixture of carrier oils may comprise plant-based oils, animal-based oils, or a combination thereof. In some embodiments, one or more carrier oils may be plant-based. Examples of plant-based carrier oils include without limitation almond oil, apricot kernel oil, avocado oil, borage seed oil, castor oil, chia seed oil, cranberry seed oil, corn oil, coconut oil, hazelnut oil, hemp oil, evening primrose oil, grapeseed oil, jojoba oil, linseed oil, macadamia nut oil, mustard seed oil, sesame oil, olive oil, cotton oil, peanut oil, pecan oil, pomegranate seed oil, poppy seed oil, rosehip oil, soybean oil, sunflower oil, and watermelon seed oil.

In some embodiments, one or more plant-based carrier oils may be an essential oil or volatile oil such as bay oil, bergamot oil, balm oil, cedarwood oil, cherry oil, cinnamon oil, clove oil, peppermint oil, and walnut oil.

In some embodiments, one or more carrier oils may be animal-based. Examples of animal-based carrier oils include without limitation poultry oil, emu oil, oleo-oil, tallow oil, fish oil, fish liver oil, and cod liver oil.

In some embodiments, one or more carrier oils may comprise one or more long chain fatty acids. Long chain fatty acids may have a straight or branched chain preferably comprising 13 or more carbon atoms. Long chain fatty acids are absorbed by the lymphatic system. Formulations comprising one or more long chain fatty acids may therefore improve absorption when combined with certain mixtures of cannabinoid(s) and carrier oil(s). In some embodiments, one or more carrier oils may comprise one or more medium chain fatty acids. Medium chain fatty acids may have a straight or branched chain preferably comprising 6-12 carbon atoms. It is possible to use one or more lengths of fatty acids in combination.

Carrier oils employed will be safe for human consumption at the dosages provided.

As described above, certain embodiments employ Camelina oil as a carrier oil. Camelina oil is widely available from commercial sources including www.threefarmers.ca.

Other embodiments employ MCT Oil as a carrier oil. MCT Oil has naturally occurring fatty acids caprylic acid (C8:0) and capric acid (C10:0). MCT Oil may be derived from coconut/palm kernel oil. MCT Oil is widely available from commercial sources including Now Foods (see https://www.nowfoods.com/sports-nutrition/mct-oil-liquid) Marine oils are preferred carrier oils in certain embodiments. Marine oils comprise oils extracted from ocean organisms including vertebrates (e.g. fish) and non-vertebrates (e.g. invertebrates, bacteria and sea-plants). A preferred carrier oil for use in the invention is oil extracted from krill. Neptune Krill Oil (NKO) has unique fatty acid profile, which includes EPA and DHA, plus phospholipids. In addition, NKO naturally has astaxanthin, a free radical scavenger and immune-supporting carotenoid. Krill oil, in particular NKO, is widely available from commercial sources including https://www.nowfoods.com/supplements/.

In some embodiments, the carrier oils comprise preferred ratios of omega-3 oils to omega-6 oils (on a weight per weight basis unless otherwise indicated). The following chart lists the omega-6 and omega-3 content of various vegetable oils and foods:

| | Omega-6 | Omega-3 |
|---|---|---|
| Oil | Content | Content |
| Safflower | 75% | 0% |
| Sunflower | 65% | 0% |
| Corn | 54% | 0% |
| Cottonseed | 50% | 0% |
| Sesame | 42% | 0% |
| Peanut | 32% | 0% |
| Soybean | 51% | 7% |
| Canola | 20% | 9% |
| Walnut | 52% | 10% |
| Flaxseed | 14% | 57% |
| Fish* | 0% | 100% |

About 75% of the fats in chia seeds consist of the omega-3 alpha-linolenic acid (ALA), while about 20% consist of omega-6 fatty acids (Ratio=3.75). The ratio for Camelina oil is 2.2 (=39/18). In some embodiments, carrier oils comprise a preferred omega-3 to omega-6 ratio (on a weight per weight basis) of about 1.0 or higher, 1.5 or higher, 2.0 or higher, or 2.2 or higher.

### Methods of Generating Lipid Carriers of the Invention

Several methods may be employed to generate an oral formulation of the invention. In some embodiments, the invention provides a method for producing an emulsion of lipid carrier(s) and cannabinoid. An emulsion may be generated simply by mixing a food grade surfactant with the lipid carrier and the cannabinoid formulation. Surfactants such as Labrasol™ are satisfactory. Gentle agitation is sufficient to generate a surfactant mixture of the invention which is an emulsion, or a surfactant mixture that becomes an emulsion upon exposure to the aqueous environment of the gastric tract. The method of generating oil-in-water emulsions generally includes the steps of (i) dissolving one or more cannabinoids and one or more lipid carriers in a polar solvent (e.g. ethanol) to obtain a cannabinoid-lipid carrier-polar solvent solution; (ii) injecting the cannabinoid-lipid carrier-polar solvent solution into distilled water and blending to obtain a cannabinoid-lipid carrier-polar solvent emulsion; and (iii) removing the polar solvent to obtain a concentrated emulsion of lipid carrier/cannabinoid. In some embodiments, the average diameter of the droplets in the emulsion is in the range of about 100 nm to 1000 nm.

In some embodiments, the invention provides a method for producing a nanoemulsion of lipid carrier(s) and cannabinoid. The method generally including the steps of (i) dissolving one or more cannabinoids and one or more lipid carrier(s) in a polar solvent (e.g. ethanol) to obtain a cannabinoid-lipid carrier-polar solvent solution; (ii) injecting the cannabinoid-lipid carrier-polar solvent solution into distilled water and blending to obtain a cannabinoid-lipid carrier-polar solvent emulsion; and (iii) removing the polar solvent to obtain a concentrated emulsion of lipid carrier/cannabinoid. In some embodiments, the average diameter of the droplets in the nanoemulsion is in the range of about 20 nm to 600 nm. In a preferred embodiment, the average diameter of the droplets in the emulsion is in the range of about 50 nm to 150 nm.

Without departing from the scope of the invention, any method may be employed to generate an emulsion or nanoemulsion. As those skilled in the art will be aware, many methods are available for generating emulsions and/or nanoemulsions, such as those found in Jaiswal, M. et al. 2015 “Nanoemulsion: An Advanced Mode of Drug Delivery System” (3 Biotech, 5(2): 123-127), and Ting, Y. et al. 2013 “Emulsion in Oral Delivery of Bioactive Lipophilic Phytochemicals” (from the book: Nutrition, Functional and Sensory Properties of Foods); see also Singh Y. et al. 2017 “Nanoemulsion: Concepts, development and applications in drug delivery” (J Control Release, 252:28-49), all of which publications are incorporated herein in their entireties.

In some embodiments, the invention provides a method for producing a liposomal formulation of lipid carrier(s) and cannabinoid. The method generally including the steps of (i) dissolving one or more lipid carrier(s) and one or more cannabinoids in a polar solvent (e.g. ethanol) to obtain a lipid carrier-cannabinoid-polar solvent solution; (ii) adding a phospholipid to the cannabinoid-lipid carrier solution to obtain a phospholipid-cannabinoid-lipid carrier solution; (iii) injecting the phospholipid-cannabinoid-lipid carrier solution into distilled water to obtain a suspension of liposomes; and (iv) removing the polar solvent from the liposomal suspension to obtain a concentrated liposomal suspension. In some embodiments, the average diameter of the liposomes in the suspension is in the range of about 50 nm to 1000 nm.

Without departing from the scope of the invention, any method may be employed to generate a liposome. As those skilled in the art will be aware, many methods are available for generating liposomes, such as those found in Alavi, M. et al. 2017 “Application of Various Types of Liposomes in Drug Delivery Systems” (Adv Pharm Bull, 7(1): 3-9), incorporated herein in its entirety.

In some embodiments, the invention provides a method for producing micellar formulations of lipid carrier(s) and cannabinoid. The method generally including the steps of (i) dissolving one or more lipid carrier(s) and one or more cannabinoids in a water miscible solvent to obtain a solution containing a mixture of lipid carrier(s) and cannabinoid; (ii) injecting the mixture into distilled water to obtain an aqueous miscible solvent suspension of micelles; and (iii) removing the water miscible solvent to obtain a concentrated micellar formulation. In some embodiments, the average diameter of the micelles in the suspension is in the range of about 50 nm to 1000 nm.

Without departing from the scope of the invention, any method may be employed to generate a micelle. As those skilled in the art will be aware, many methods are available for generating micelles, such as those found in Xu, W. et al. 2013 “Polymeric Micelles, a Promising Drug Delivery System to Enhance Bioavailability of Poorly Water-Soluble Drugs” (J Drug Deliv, 340315), incorporated herein in its entirety.

In some embodiments, the methods of the present invention may include one or more additional steps. An additional step may include (a) adding a stabilizer, carrier, buffer, excipient, or the like, to a formulation of the present invention; (b) encapsulating a formulation of the present invention. Encapsulation of cannabinoid-lipid carrier formulations can take place by trapping or dispersing cannabinoid-lipid carrier formulations within an encapsulating matrix. In some embodiments, encapsulation involves trapping cannabinoid-lipid carrier formulations within a network of crosslinked polymers. In some embodiments, encapsulation involves trapping cannabinoid-lipid carrier formulations within a network of non-crosslinked polymers. In some embodiments, encapsulation involves dispersing cannabinoid-lipid carrier formulations within the crystalline structure of a macromolecule. Examples of crystalline structure-forming macromolecules include without limitation sugars, starches, and proteins.

### Pharmaco-Analytical Testing of Cannabinoid(s) for Use in Preparation of the Defined Dose Oral Formulations

Any chemical analytical method may be employed to determine the amount of the cannabinoids in the preparation used for formulating the UDF. Many methods are available to those skilled in the art, such as those found in Thomas, B F and El Sohly, M 2015 “The Analytical Chemistry of Cannabis: Quality Assessment, Assurance, and Regulation of Medicinal Marijuana and Cannabinoid Preparations” (Elsevier). See also Wang et al. (2016) Decarboxylation Study of Acidic Cannabinoids: A Novel Approach Using Ultra-High-Performance Supercritical Fluid Chromatography/Photodiode Array-Mass Spectrometry. Cannabis Cannabinoid Res.; 1(1): 262-271; and Wang et al. (2017) Quantitative Determination of Cannabinoids in Cannabis and Cannabis Products Using Ultra-High-Performance Supercritical Fluid Chromatography and Diode Array/Mass Spectrometric Detection. J Forensic Sci.; 62(3):602-611.), all of which are incorporated herein in their entireties.

Testing may be performed to identify the cannabinoid content of the ground dried plant form, any other solid form or a liquid extract preparation.

Testing may be required at one step or at multiple steps in the production process. It may be first performed as a batch assay to ascertain amounts of relevant cannabinoids from a particular harvest or extraction process. The representative sample and measurement technique must be sufficient to represent all samples of the process batch within the degree of variability tolerated by the overall process, namely +/−25% of the defined dose of each cannabinoid. Depending on the result, the cannabinoid preparation may need to be adjusted (either diluted or concentrated) to generate a cannabinoid preparation to meet the tolerance range of volume/dose range for manufacturing specifications of the UDF. The operator will have available a variety of cannabinoid diluents or concentrating processes and/or oils of known cannabinoid concentrations to adjust the preparation. Often only one cannabinoid will need to be added, the others being already at satisfactory levels. The operator can determine by simple algorithm the amount of which additives and/or which concentration steps are required to obtain the desired preparation. The final preparation of cannabinoid may again be chemically analyzed. Any final preparation which is not within tolerance levels is discarded or re-processed until desired cannabinoid levels are obtained. The final tolerance level is within +/−25%, preferably within +/−20%, +/−15%, +/−10%, +/−5%, +/−2% and most preferably within +/− about 1% of the desired in-going amount of each defined dose cannabinoid in the preparation used for formulating the UDF.

Alternatively stated, the UDF is expected to contain a dose of from 80% to 120% of the amount stated on product label. Preferably the range will be significantly more precise.

### Analytical Identification of Terpenes and Other Cannabis Plant Components in the Cannabinoid Preparation

Depending on the extraction process employed, a variety of other plant constituents may be extracted from cannabis along with the cannabinoids. It may be desirable to identify and confirm concentrations of these components. Terpenes, chlorophylls, other alkaloids and macromolecules may also be detected by gas chromatography, mass spectroscopy, high-pressure liquid chromatography, or techniques standard in the art.

In certain embodiments, the signifier used with the unit dosage form product may also indicate the defined dose of such additional plant components.

### Coatings and Excipients

Oral formulations of the invention may optionally further comprise additional components such as but not limited to carrier oils, stabilizers, anti-oxidants, preservatives and excipients, as further described below.

Excipients may be used for the purpose of stabilizing cannabinoids of the oral dosage forms, or to confer a therapeutic enhancement such as facilitating drug absorption, reducing viscosity, or enhancing solubility. Useful excipients include without limitation diluents, dissolving agents, solubilising agents, adjuvants, anti-adherents, binders, coatings, colorants, surfactants, bioadhesives, polysaccharides, polymers, copolymers, bioavailability enhancing agents, mucoadhesives, protective agents, buffers, antioxidants, dispersing agents, lubricants, sorbents, preservatives, flavor imparting agents, and any combination thereof. Excipients may include one or more pharmaceutically acceptable carriers, diluents, fillers, hinders, lubricants, glidants, disintegrants, bulking agents, flavourants or any combination thereof. Non-limiting examples of suitable pharmaceutically acceptable carriers, diluents or fillers for use in the invention include lactose (for example, spray-dried lactose, alpha-lactose, beta-lactose), or other commercially available forms of lactose, lactitol, saccharose, sorbitol, mannitol, dextrates, dextrins, dextrose, maltodextrin, croscarmellose sodium, microcrystalline cellulose (for example, microcrystalline cellulose available under the trade mark Avicel), hydroxypropylcellulose, L-hydroxypropylcellulose (low substituted), hydroxypropyl methylcellulose (HPMC), methylcellulose polymers (such as, for example, Methocel A, Methocel A4C, Methocel A15C, Methocel A4M), hydroxyethylcellulose, sodium carboxymethylcellulose, carboxymethylene, carboxymethyl hydroxyethylcellulose and other cellulose derivatives, pre-gelatinized starch, starches or modified starches (including potato starch, corn starch, maize starch and rice starch) and the like. Typically glidants and lubricants may also be included in the invention. Non-limiting examples include stearic acid and pharmaceutically acceptable salts or esters thereof (for example, magnesium stearate, calcium stearate, sodium stearyl fumarate or other metallic stearate), talc, waxes (for example, microcrystalline waxes) and glycerides, light mineral oil, PEG, silica acid or a derivative or salt thereof (for example, silicates, silicon dioxide, colloidal silicon dioxide and polymers thereof, crospovidone, magnesium aluminosilicate and/or magnesium alumina metasilicate), sucrose ester of fatty acids, hydrogenated vegetable oils (for example, hydrogenated castor oil), or mixtures thereof or any other suitable lubricant. Suitably one or more binders may also be present in the invention and non-limiting examples of suitable binders are, for example, polyvinyl pyrrolidone (also known as povidone), polyethylene glycol(s), acacia, alginic acid, agar, calcium carragenan, cellulose derivatives such as ethyl cellulose, methyl cellulose, hydroxypropyl cellulose, hydroxypropyl methyl cellulose, sodium carboxymethylcellulose, dextrin, gelatin, gum arabic, guar gum, tragacanth, sodium alginate, or mixtures thereof or any other suitable binder. Suitable disintegrants may also be present in the invention. Examples include, but are not limited to, hydroxylpropyl cellulose (HPC), low density HPC, carboxymethylcellulose (CMC), sodium CMC, calcium CMC, croscarmellose sodium; starches exemplified under examples of fillers and also carboxymethyl starch, hydroxylpropyl starch, modified starch; crystalline cellulose, sodium starch glycolate; alginic acid or a salt thereof, such as sodium alginate or their equivalents and any combination thereof.

The term “antioxidant” as used herein includes any compound or combination of compounds that prevent or slow down oxidation of components caused by the damaging reactive oxygen species (ROS). Any of the known antioxidants may be used, including but not limited to tocopherols, phospholipids (PL), phytosterols, phycocyanin, vitamins E, A and C, betacarotene, coenzyme Q10, fatty acids omega-3, omega-6 and w-9, phytoantioxidants such as polyphenols, terpenes as butylated hydroxyanisole (BHA), butylated hydroxytoluene (BHT), propyl gallate, lecithin, sesamin, sesamol, sesamolin, α-tocopherol, γ-tocopherol, salicylic acid, ascorbic acid, ascorbyl palmitate, fumaric acid, malic acid, sodium ascorbate and sodium meta-bisulphite, as well as chelating agents such as disodium EDTA. Pharmaceutically acceptable nutraceutical dietary supplements may also be employed as anti-oxidants including plants, alga, and lichen and may include one or more extracts of honeybee propolis, red clover, soybean, caper, almond, milk thistle, green tea, pomegranate, orange red, grape seed, bilberry, foti root, ginseng, english ivy, red algae, brown algae, green algae and lichens.

The total moisture (water) content of the UDF may be selected to ensure appropriate stability and shelf-life for the product. Those skilled in the art may identify acceptable ranges depending on the form of UDF selected. Softgels are particularly sensitive to water content as water will weaken and dissolve softgel gelatin capsules. Water content is typically kept below 30% and preferably below 5% of the total mass.

In preferred embodiments, certain potential contaminants are eliminated, avoided, or present at trace levels considered acceptable for human consumption. In particular, the preferred embodiments eliminate, avoid or reduce the presence of organic solvents, pest control products, di-acetyl and ammonia.

In some embodiments, at least one emulsifying agent is used to improve the stability of the cannabinoid active ingredients in oral formulations of the present invention. The stability of an emulsion refers to the ability of the emulsion to resist change in its properties over time. An emulsifying agent may be used to modify a hydrophobic cannabinoid oil so as to enhance hydrophilicity such that the hydrophobic cannabinoid oil is more bio-available. Examples of emulsifying agents that are safe for human consumption include without limitation, Labrasol™ xanthan gum, guar gum, alginate, cyclodextrin, lecithin, cellulose, hyaluronic acid, carrageen, monoglycerides, natural emulsifiers, organic emulsifiers.

In some embodiments, oral formulations of the present invention may be formulated with modified- or sustained-release coatings or layers. Modified- or sustained-release coatings are designed to delay release of active cannabinoids for a certain period of time, to target release to a certain area of the body, or to maintain a constant drug concentration for a specific period of time with minimal side effects. Controlled release can be achieved by coating UDFs using various pharmacological technologies. For example, poly(meth)acrylate coatings have been widely used in the pharmacological industry to achieve targeted and controlled drug release. Other suitable enteric coatings include without limitation hydroxypropyl methylcellulose, poly(meth)acrylates, methyl acrylate-methacrylic acid copolymers, cellulose acetate, cellulose acetate phthalate, polyvinyl acetate phthalate, plastics plant fibres, and other types of coatings.

In some embodiments, enteric coatings may be used to delay release of cannabinoids until they reach the intestine. An enteric coating that dissolves at a pH of about 6.0 to 7.0 may be used for this purpose. An example of a suitable enteric coating that dissolves in the small intestine but not in the stomach is cellulose acetate phthalate.

In some embodiments, a long-chain fatty acid of specific length shown to be best with the specific cannabinoid or cannabinoids of interest, combined with a specific micellar particle size may be used.

In some embodiments a long-chain fatty acid of specific length shown to be best with the specific cannabinoid or cannabinoids of interest, combined with a specific micellar particle size combined with enteric coatings designed to place the release of the dosage form to a specific part of the GI tract may be used.

In a preferred embodiment, the UDF meets the requirements of a dissolution or disintegration test that is applicable to its formulation and that is set out in European Pharmacopoeia, The Canadian Formulary, The United States Pharmacopoeia, and/or The Pharmaceutical Codex: Principles and Practices of Pharmaceuticals, incorporated herein in its entirety.

### Bioavailability

A significant drawback to the use of cannabinoid-based products is the lack of a consistent and robust method of delivery. The route of administration and formulation can significantly affect the rate of cannabinoid absorption. For example, studies show that the bioavailability of THC from inhaled cannabis smoke can fluctuate between 2-56% compared to 4-20% for orally consumed cannabis (Huestis 2007; PMCID: PMC2689518 and McGilveray 2005; PMID: 16237477, both incorporated by reference in their entireties).

Smoking can provide a rapid and efficient method of drug delivery from the lungs to the brain but these advantages are offset by the harmful effects of cannabinoid smoke, which are not appealing to many users for health or lifestyle reasons. Oral delivery is therefore a common alternative route of administration for cannabinoid products, particularly because cannabinoids can be easily incorporated into food and beverage products or ingested in tablet, pill, capsule, or similar forms.

Absorption of cannabinoids can be slower when ingested, with lower and sometimes delayed peak cannabinoid concentrations (Ohlsson 1981; PMID: 6261133), incorporated herein in its entirety. Dose, vehicle, and physiological factors such as rates of metabolism and excretion can influence cannabinoid concentrations in circulation. Variable absorption, degradation of cannabinoids in the stomach, and first-pass metabolism to active and inactive metabolites in the liver are the main reasons for the low oral bioavailability of cannabinoids. Effective oral formulations of cannabinoids are therefore essential to maximize their therapeutic effects.

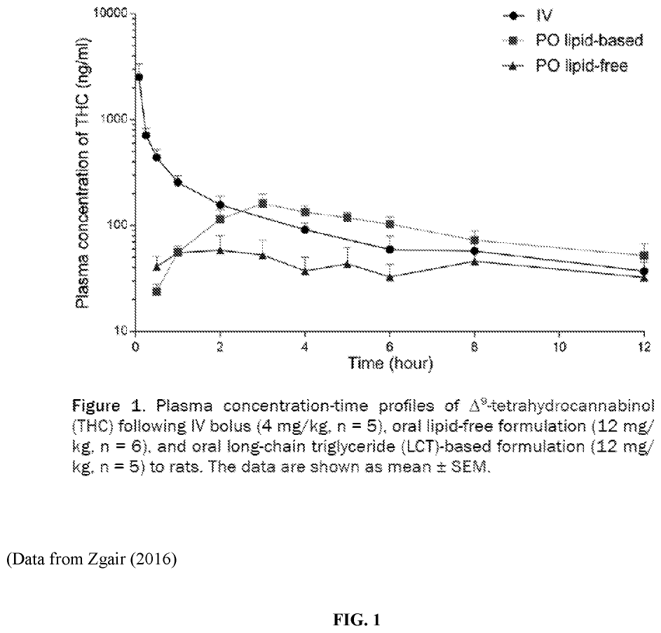

An advantage of the invention is the improved bioavailability of the claimed oral formulations. Historical long chain triglycerides such as sesame oil and other vegetable oils have been added to formulations to help dissolve the lipid soluble cannabinoids The potential effect of sesame oil (mostly comprised of long-chain triglycerides (LCT)) on improving bioavailability of oral cannabinoid doses, examining only THC and CBD, was reported in Zgair et al. (2016) Am J Transl Res 2016; 8(8):3448-3459. Specifically, the AUC and Cmax of lipid-based formulations were enhanced over lipid-free formulations (c.f. Zgair FIG. 1/Table 3 (for THC)).

The relative bioavailability of cannabinoid formulations provided herein was determined by assessing their pharmacokinetic profiles using well known techniques such as area under the curve (AUC; which is a measure of the overall exposure of a subject to the cannabinoid, or of a liver metabolite thereof, in the plasma after a dose), Cmax (i.e. the highest concentration of cannabinoid, or a liver metabolite thereof, in the plasma that is measured after a dose) and Tmax (ie. the time after administration of a drug when the maximum plasma cannabinoid concentration is reached)—all of these measurements are extensively described in the art.

The oral formulations of the present invention provide enhanced AUC and Cmax of the cannabinoids delivered in comparison both to lipid free formulations and/or to the LCT formulations reported by Zgair, supra. They also provide formulations not previously investigated of other cannabinoids of interest and other lipid carriers of the identified carbon chain lengths and modifications. Formulations provided herein may optionally employ the formulation techniques, including micellar formulations, disclosed herein, and optionally with coatings specifically designed to place the capsules/tablets in a specific section of the GI tract before dissolution.

In a preferred embodiment, the invention comprises a cannabinoid oil extract with a carrier oil selected from MCT oil, camelina oil and a marine oil, forming particles of 20-600 nm diameter (95% distribution) and encapsulated in a gel capsule (hardgel or softgel). In an alternative embodiment, such capsule is coated with delayed release coating. In further preferred embodiments the delayed release coating is triggered to resist release in the acid conditions of the stomach and to provide enhanced release elsewhere in the gut such as the small intestine (pH 6), the terminal ileum (pH 7.4) or the caecum (pH 5.7).

FIG. 1 graphs the bioavailability of THC absorbed from the LCT formulation (12 mg/kg THC in sesame oil) reported by Zgair supra. Sesame oil is an LCT oil. It has high levels of Omega-6 (ω6) and low levels of Omega-3 (ω3), corresponding to a: ω3:ω6 ratio of approximately 1:50 (0.02).

### General UDF Production Methods

Having selected the amounts and concentrations of all ingredients of the oral formulation of the invention, the ingredients will be formulated together for preparing the unit dosage form. Those skilled in the art are familiar with identifying preferred formulation techniques for the UDF. In a preferred embodiment, the UDF is a pill, tablet, capsule, film, or wafer, any of which may optionally be orally disintegrating, or a lollipop, lozenge, oil, tincture, or syrup. The formulation process will be adjusted accordingly. Pills and tablets are prepared from solid formulations. Syrups, oils and tincture are liquid formulations. An orally disintegrating film, wafer, tablet or a lollipop or lozenge provides the UDF in an oral form wherein the active ingredients are at least partly absorbed directly in the buccal cavity. Capsules may be either solid formulations (e.g. powders or particles in a hard-gel) or liquid formulations (e.g. oil-based formulations used in soft-gels). Oil based formulations with little or no water are typically easily encapsulated. Oil-in-water formulations may comprise microemulsions, liposomes, nanoemulsions, micelles, and other forms known in the art.

Preferred capsule types are soft gelatin capsules (softgels) and hard gelatin capsules. Soft Gelatin Capsules (softgels) are well known in the art. Typically soft-gels are used for formulations not based on water, such as oil-based solutions, because water based solutions would dissolve the gelatin. The basic steps of softgel manufacturing are: Gelatin Preparation (the process of blending and heating granulated gelatin into a thick syrup for use in encapsulation); Fill Material Preparation (the process of preparing the non-aqueous oil or paste containing the lipid carrier and cannabinoid components that will be encapsulated); Encapsulation (the process of converting the gel mass into a thin layer of gelatin and wrapping it around the fill material to form a softgel); Drying (the process which removes excess moisture from the gelatin shell to shrink and firm up the softgel); the softgel could incorporate a Coating step (the process of coating the capsule with a coating designed to release the capsule within the digestive system); and finally Cleaning, Inspection and Sorting. Automated or semi-automated manufacturing of softgels and can be achieved using commercially available equipment, such as that provided by CapPlus Technologies, SaintyCo, and many others. Hard gelatin capsules are made of two parts, the body and a cap. This form of capsule holds dry ingredients in the form of powders, granules or tiny pellets. They may also include cannabis oils of various viscosity, such as diluted cannabis oil and concentrated cannabis extracts. The body is first filled with the mix of active ingredients and any excipients used, and then closed with the cap using either a manual or automated or semi-automated capsule filling machine, such as those commercially available from Bosch, Zanazzi, etc. Banding of hard gelatin capsules is sometimes useful to prevent leakage.

The inventors recognize that advantages may be achieved by use of a dose form that is substantially opaque to one or both of ultraviolet and visible light, such as a photo- and/or UV-opaque gelatin capsule. A general form of this technology is described in co-owned patent application U.S. Ser. No. 62/837,848 filed 24 Apr. 2019, incorporated herein by reference in its entirety.

Delayed release to the gastrointestinal tract can be achieved for softgels or hard gels by enteric coatings which delay disintegration until after passing from stomach to the intestine; or by formulation techniques such as pellets which resist release until they pass into a specific intestinal domain. Such techniques are widely known in the art. An example is WIPO patent publication WO2017075215A1 to McGuffy and Bell, incorporated herein by reference in its entirety, for extended release film-coated softgel or hard-shell capsules.

A wide variety of technologies are available for a buccal or sublingual formulation such as an orally disintegrating thin film, wafer or tablet, or a lollipop, and/or lozenge. Sublingual tablets, wafers, films and strips can be designed to rapidly disintegrate (5-15 seconds) providing rapid access to buccal cavity capillaries and avoid the hostile environment of the gastrointestinal track. Lollipops and lozenges provide a combination of buccal and gastric administration. The technologies are widely used with therapeutic agents where rapid onset is desired. (See Lamey and Lewis “Buccal and Sublingual Delivery of Drugs” Ch 2 in “Routes of Drug Administration” Ed. Florence and Salole (Butterworth-Heinemann). Orally disintegrating tablets (ODTs) of the invention can be based on solid lipid micro-pellets (SLMPs). SLMPs can be prepared using the hot melt extrusion technique and utilizing lipid carriers such as Compritol, Precirol and white beeswax either alone or in mixtures. Such ODTs demonstrate sustained drug release and a taste masking effect.

Association with Signifier

In preferred embodiments, methods and compositions of the invention may associate the disclosed formulations with one or more signifiers permitting the consumer to determine the defined dose of selected cannabinoids therein. A “signifier” means a visual mark or symbol that the consumer recognizes as referring to a specific defined dose. The signifier chosen may have elements of meaning, such as a number and unit, (e.g. “5 mg” or “10 mg” or simply “5” or “10”) or it may be an abstract signifier, where its meaning, in terms of defined dose, can be determined by reference to a standard. The meaning may be determined directly by the consumer or indirectly via a device.

The signifier may be associated directly with the formulation by such means as embossing, or by colour, pattern or shape feature. Alternatively, the signifier may be associated with the packaging. The packaging may include signifiers directly interpreted by consumers or signifiers which are machine readable codes. In all embodiments, the signifier allows the consumer to determine the defined dose of selected cannabinoids therein.

The signifier may be associated directly with the UDF before, during or after encapsulation by such means as edible ink(s) imprinted on the surface of the capsule, or by embossing, by engraving (such as laser-engraving), or by color, pattern or shape feature. The edible ink applied to the capsule may include shellac from about 10% to about 30% by weight, about 20% to 70% by weight of at least one solvent, and at least one soluble or insoluble pigment from about 10% wt to about 40% wt. The shellac provides structure, enhances adherence to the printing plate and capsule, and/or act as a pigment carrier. An edible ink formulation may include 10% wt to about 30% wt shellac.

### Packaging

After a UDF is manufactured, storage and delivery to consumer may be provided by:
a. Packaging the UDF individually in a blister pack; or
b. Packaging multiple UDFs in a re-sealable package.

The UDF is preferably provided in a sealed package, which functions as a barrier limiting moisture fluctuation, reducing oxidation, and enhancing shelf-life, etc. The packaging is optionally a gas-impermeable container having a hermetic closure which in the context of the present invention includes a blister pack. The UDFs may be individually sealed and packaged in blister packs. The blister packs may be designed to be child resistant and/or senior friendly in order to increase safety and convenience. While physically protecting the UDFs, the blister pack controls humidity and is impermeable to gas exchange thereby enhancing shelf life.

Examples of the substantially gas exchange impermeable packaging include, but are not limited to, A1/A1 blister, and A1-polychloro-3-fluoroethylene homopolymer/PVC laminate blister. Alternatively, the sealed package may be a re-sealable multi-package impermeable to gas exchange.

UDFs of the invention may be expelled from production into the open blister cavities. Cavity depth and shape must be suitable for the unit. The open blister cavity is then sealed with a gas impermeable membrane to maintain quality of product and to reduce dehydration, rehydration or oxidation. To eliminate oxidation altogether, the packaging may be performed in an inert gas atmosphere. Optionally the blister is packed in an inert gas atmosphere such as nitrogen gas comprising little or no oxygen. To achieve this objective, the final sealing step of the packaging method may be operated in the inert gas atmosphere in a gas enclosure protected from ambient air.

### EXAMPLES

### Example 1: Use of Lipid Carriers to Enhance Cannabinoid Bioavailability

Oral formulations of the invention are tested to determine key pharmacokinetic (PK) parameters and to identify which lipid carriers provide enhanced exposure over time (bioavailability). PK assays are used to identify plasma concentration over time, area under the curve (AUC) exposure over 24 hrs, systemic clearance rate (CL) and systemic bioavailability. The combination is also tested against the individual components. The 24 hr exposure identifies if the UDF should be administered QD (once a day) or BID (×2 a day) or more often, or less often.

Standard PK models are widely available and can be performed with a commercial service. A preferred method is to use at least 4 Male Sprague Dawley rats (210-230 g) who receive either an intravenous (i.v. 2, 5, 10 mg/kg) or oral (5, 10, 12, 20 and/or 30 mg/kg) dose of each compound separately, or combined in formulation. Blood, urine, cerebrospinal fluid (CSF) or other appropriate biological fluid is removed at periodic intervals. The biological fluid is tested for active compound(s) in order to construct concentration vs. time profiles. These data are analyzed and pharmacokinetic parameters are calculated in order to assess in vivo pharmacokinetic activity.

The study uses a fixed dose of each component in the combination in a fixed vehicle formulation. In one embodiment PEG (polyethylene-glycol) is an excipient, or alternatively a long chain fatty acid oil carrier. Typically, components are prepared from a powder form, first in 5% ETOH, then with 40% PEG. The components are combined and topped up with distilled water to 100% volume. If components are not sufficiently soluble, PEG may be increased to 60% and also add 10% PG. By way of example, a PK study may be conducted at 10 mg/kg dose for an oral formulation. 2 mg/kg may be used for IV injection as a comparison. In either case, plasma samples are collected over a 24 hr time-period to determine bioavailability. Plasma samples are tested by HPLC or LC-MS/MS to obtain PK parameters. 4 rats per each route (total n=6) is typically sufficient.

Plasma and other tissue samples are tested for the administered cannabinoids and the lipid carrier administered to the animal. The samples are also tested for significant metabolites, some of which may have more potent effects than the parent administered compounds. The samples may also be used to determine baseline levels of serum biomarkers which are relevant to the development or treatment of the complex disease models set out further below. Many serum biomarkers are of great interest in the development or treatment of complex disorders. Biomarkers of interest to the compositions of the invention include IL-6, NFkB, TNFalpha, C-reactive protein, and any other biomarker known to be or potentially implicated in the development of a disease or disorder.

### Example 2: Comparison of Carrier Oils

The experiment of Example 1 was performed with the following modifications: THC was prepared by Emerald Health Therapeutics (Victoria, BC) in ethanol-extracted cannabis concentrates diluted in each of a selection of carrier oils including: sesame oil (Sigma-Aldrich, SKU #S3547), krill oil (Grizzly Pet Products, SKU #B00VEZHL7K), camelina oil (Neptune Wellness Solutions, Product Code: RCAMO001) and camelina oil with Labrasol™ surfactant (Gattefossé, Product Code: 160865). The latter camelina oil and Labrasol™ surfactant formulation was prepared by slowly adding Labrasol™ to camelina oil heated to 60-70° C. The mixture was gently agitated and THC extract slowly added, followed by further gentle agitation to ensure THC is fully solubilized. For the oral-administered groups THC was formulated to a dose of 12 mg/kg in the carrier oil. The volume of administration is adjusted per individual animal's body weight.

Rats are orally administered one of the above formulations by oral gavage (volume up to 0.5 ml) at time zero. 0.25 ml blood was collected from the arterial catheter at 60, 180, 300, 480, 720 and 1440 minutes after drug treatment. Blood samples were promptly centrifuged to separate plasma, stored in duplicate and kept at −80° C. until ready for analysis.

Blood samples were purified and analyzed with LCMS for measurement of cannabinoid levels. Each sample was measured for levels of THC, 11-OH-THC, and THC—COOH (also known as 11-Nor-9-carboxyTHC, this compound is a non-psychoactive liver metabolite of THC with a long half-life). Pharmacokinetic parameters, including Cmax and AUC, were calculated by PK Solver, as described in Zhang et al. Comput. Methods Programs Biomed., 2010; 99(3):306-314

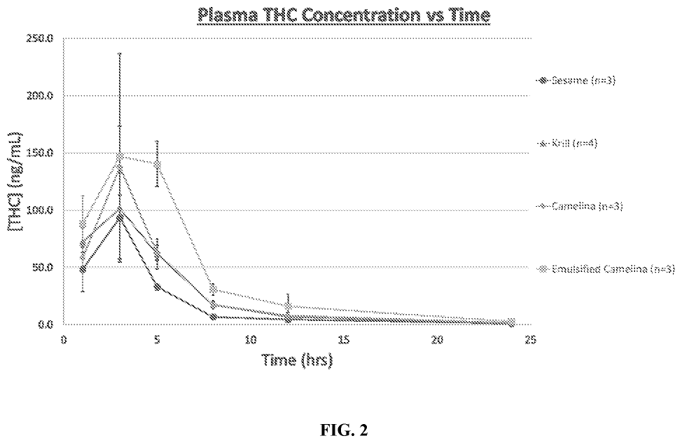

FIG. 2 sets out observed results of the experiment. The THC and emulsified camelina oil combination showed increased THC bioavailability (Cₘₐₓ=147 ng/ml; AUC₀₋ₜ=1028.2 ng/ml*hr−) at all timepoints versus the other formulations (FIG. 2). The non-emulsified camelina oil formulation also showed increased THC bioavailability (Cₘₐₓ=137.6 ng/ml; AUC₀₋ₜ=651.0 ng/ml*hr) over the krill oil (Cₘₐₓ=101.7 ng/ml; AUC₀₋ₜ=588.8 ng/ml*hr) and sesame oil (Cₘₐₓ=93.7 ng/ml; AUC₀₋ₜ=407.9 ng/ml*hr) formulations.

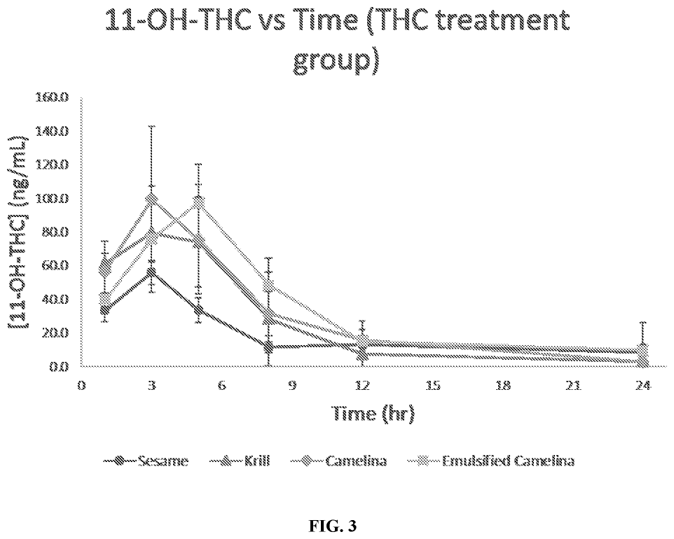

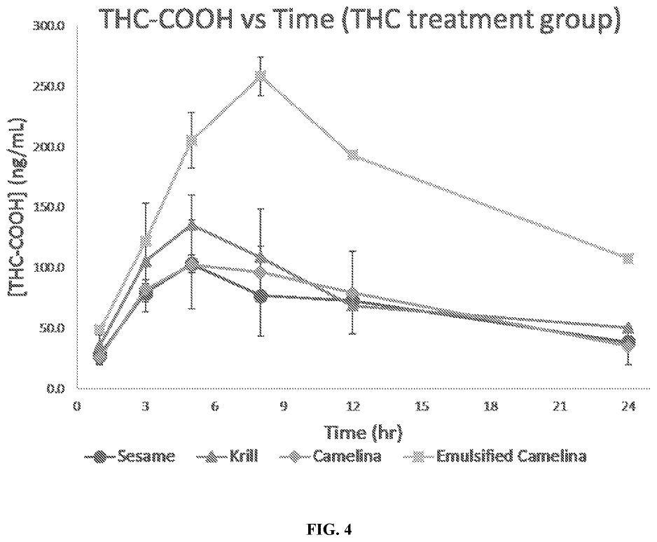

Increased THC bioavailability with both the emulsified and non-emulsified camelina formulations, and the krill formulation was further supported by observed increases in THC liver metabolites 11-OH-THC (FIG. 3) and THC—COOH (FIG. 4) as compared to the sesame oil formulation.

Taken together, the results show that camelina oil provides a significant enhancement to bioavailability of THC taken orally. This effect is particularly enhanced when the camelina is formulated with a surfactant such that it becomes an emulsion upon administration to the aqueous gut environment. Krill oil also provides an enhanced bioavailability compared to sesame oil. Camelina oil and krill oil are examples of carrier oils having a relatively high ratio of Omega-3 to Omega-6 lipids of about 1.0 or greater. These observations lead the inventors to conclude that such formulations will provide significantly different physiological effects compared to THC products taken with Omega-3/Omega-6 ratios below 1.0.

Thus, in certain embodiments, THC is provided in camelina oil, preferably mixed with surfactant, where with THC effects on the consumer are substantially enhanced compared to THC without a carrier oil or surfactant. Alternative preferred products, include lower dose THC capsules (e.g. 4 mg THC) which are provided in a carrier oil having a ratio of Omega-3 to Omega-6 lipids of about 1.0 or greater, such as a camelina oil emulsion, which due to enhanced bioavailability, have the effect of 10 mg THC capsules provided without carrier oil, or with a long chain fatty acid carrier such as sesame oil which has a low ratio of Omega-3 to Omega-6 lipids of below 1.0.

These examples are derived from the observations presented in FIGS. 2-4 that the AUC and Cmax of the camelina emulsion formulation is significantly greater, for the same dose, as the sesame formulation.

Summarized otherwise, the invention can be described as a method to enhance the effect of THC to a consumer by providing it in a carrier oil having a high ratio of Omega-3 to Omega-6 lipids of about 1.0 or greater, and optionally with such oil in an oil carrier emulsion, most preferably where the carrier oil is camelina oil. Another description is a “high-impact THC capsule” where a lower dose THC capsule according to the invention has the physiological effect of a higher dose prior art capsule. In some embodiments, the invention provides a physiological impact of about 2.5 times higher than a prior art dose of THC in a prior art formulation.

The results of FIG. 1 are summarized in the following Table 2.

TABLE 2

| | | | % C_{Max} | | % AUC₀₋ₜ |
|---|---|---|---|---|---|
| | | | (relative to | AUC₀₋ₜ | (relative to |
| | | Cₘₐₓ | sesame oil | (ng/mL * | sesame oil |
| Cannabinoid | Carrier Oil | (ng/mL) | control group) | hr) | control group) |
| THC | Sesame | 93.7 | 100.0% | 407.9 | 100.0% |
| | Krill | 101.7 | 108.5% | 588.8 | 144.3% |
| | Camelina | 137.6 | 146.9% | 651.0 | 159.6% |
| | Emulsified Camelina | 147.0 | 156.9% | 1028.2 | 252.1% |

The results of FIG. 2 are summarized in the following Table 3.

| | | | % C_{Max} | | % AUC₀₋ₜ |
|---|---|---|---|---|---|
| | | | (relative to | AUC₀₋ₜ | (relative to |
| THC | | Cₘₐₓ | sesame oil | (ng/mL * | sesame oil |
| Metabolite | Carrier Oil | (ng/mL) | control group) | hr) | control group) |
| 11-OH-THC | Sesame | 56.0 | 100.0% | 424.3 | 100.0% |
| | Krill | 79.5 | 142.0% | 583.3 | 137.5% |
| | Camelina | 99.4 | 177.5% | 699.5 | 164.9% |
| | Emulsified Camelina | 97.5 | 174.1% | 785.0 | 185.0% |

The results of FIG. 3 are summarized in the following Table 4.

| | | | % C_{Max} | | % AUC₀₋ₜ |
|---|---|---|---|---|---|
| | | | (relative to | AUC₀₋ₜ | (relative to |
| THC | | Cₘₐₓ | sesame oil | (ng/mL * | sesame oil |
| Metabolite | Carrier Oil | (ng/mL) | control group) | hr) | control group) |
| THC-COOH | Sesame | 103.4 | 100.0% | 1533.0 | 100.0% |
| | Krill | 135.5 | 131.0% | 1819.5 | 118.7% |
| | Camelina | 102.8 | 99.4% | 1632.2 | 106.5% |
| | Emulsified Camelina | 258.4 | 249.9% | 3904.5 | 254.7% |

Product embodiments that take advantage of these surprising properties are set out in Example 3.

### Animal Models of Complex Disease or Condition

Compositions of the invention are tested in models corresponding to the disease and/or conditions proposed for use. These may be selected from among models of anxiety, pain, sleep induction, calmness induction, alertness induction, weight control, weight loss, obesity, diabetes and metabolic syndrome.

For any of the animal assays herein (including human testing), successful treatment may be identified according to the behavioural results identified in the assay, or by measuring biomarkers of disease progression/treatment, such as IL-6, NFkB, TNFalpha, C-reactive protein, and any other biomarker known to be or potentially implicated in the development of the disease or disorder being studied. Those skilled in the art are familiar with the wide variety of models available for further testing the products of the invention.

### Example 3: Unit Dosage Form (UDF) Oral Capsule Embodiments

### Example 3-1—Island Mist Capsule (HardGel; Low-Dose; 250 mg Volume)

| Ingredient | Amount | |
|---|---|---|
| CBD | 25 | mg |
| THC | 1 | mg |
| Other cannabis extract ingredients: | 19 | mg |
| lipids, waxes, sugars, and other | | |
| phytochemicals and anti-oxidants | | |
| Krill oil (amount estimated | 205 | mg |
| based approximate fill volume) | | |
| Net weight of capsule contents | 250 | mg |
| Capsule (HardGel Size 3) | 48 | mg |
| Gross weight of capsule (estimated) | 298 | mg |

### Example 3-2—Island Mist Capsule (SoftGel; High-Dose CBD; 250 mg Volume)

| Ingredient | Amount | |
|---|---|---|
| CBD | 100 | mg |
| THC | 4 | mg |
| Other cannabis extract ingredients: | 84 | mg |
| lipids, waxes, sugars, and other | | |
| phytochemicals and anti-oxidants | | |
| Camelina Oil (amount estimated | 62 | mg |
| based on approximate fill volume) | | |
| Net weight of capsule contents | 250 | mg |
| Capsule (SoftGel Size 5) | 48 | mg |
| Gross weight of capsule (estimated) | 298 | mg |

### Example 3-3—TimeWarp A3 CAPSULE (Softgel; Low-Dose TUC; 250 mg Volume)

| Ingredient | Amount | |
|---|---|---|
| CBD | 0 | mg |
| THC | 10 | mg |
| Other cannabis extract ingredients: | 8 | mg |
| lipids, waxes, sugars, and other | | |
| phytochemicals and anti-oxidants | | |
| Camelina Oil (amount estimated | 232 | mg |
| based on approximate fill volume) | | |
| Net weight of capsule contents | 250 | mg |
| Capsule (SoftGel Size 5) | 48 | mg |
| Gross weight of capsule (estimated) | 298 | mg |

### Example 3-4—Island Mist Capsule (HardGel; Low-Dose THC; Medium Dose CBD; 250 mg Volume)

| Ingredient | Amount | |
|---|---|---|
| CBD | 60 | mg |
| THC | 2.5 | mg |
| Camelina oil/Labrasol ™ surfactant | 187.5 | mg |
| (95:5 ratio by weight) | | |
| Net weight of capsule contents | 250 | mg |
| Capsule (HardGel Size 3) | 48 | mg |
| Gross weight of capsule (estimated) | 298 | mg |

Certain preferred embodiments of the invention are set out in Table 5

TABLE 5
Preferred embodiments of the invention

| | | | | | | | | | | Final | |
|---|---|---|---|---|---|---|---|---|---|---|---|
| | | | Amount | | | | | | | Capsule | |
| Prod | | | Oil | Defined Dose (mg) | | | | | | Mass* | |
| # | Capsule | Oil | (mg) | THC | THCA | THCV | CBD | CBG | CBC | (mg) | Indications |
| 3-1 | HardGel | MCT | 205 | 1 | 9 | | | | | 432 | Pain, Nausea, |
| | | | | | | | | | | | Obesity, |
| | | | | | | | | | | | Metabolic |
| | | | | | | | | | | | Syndrome, |
| | | | | | | | | | | | Diabetes, |
| | | | | | | | | | | | Inflammation |
| 3-2 | HardGel | Camelina | 62 | 10 | | | | | | 432 | Pain, Appetite |
| | | | | | | | | | | | enhancement, |
| | | | | | | | | | | | Inflammation; |
| 3-3 | HardGel | Camelina | 232 | 10 | | | 10 | | | 577 | Pain; Anxiety; |
| | | | | | | | | | | | Sleep, |
| | | | | | | | | | | | Inflammation; |
| 3-4 | HardGel | MCT | 141 | 10 | | | 10 | | | 777 | Pain; Anxiety; |
| | | | | | | | | | | | Sleep, |
| | | | | | | | | | | | Inflammation; |
| 3-5 | SoftGel | Camelina | 420 | 10 | | | 10 | | | 500 | Pain; Anxiety; |
| | | or krill | | | | | | | | | Sleep, |
| | | | | | | | | | | | Inflammation; |
| 3-6 | SoftGel | Camelina | 420 | 1 | | | 25 | | | 500 | Anti-epileptic, |
| | | or krill | | | | | | | | | Chronic Pain, |
| | | | | | | | | | | | Inflammation, |
| | | | | | | | | | | | Schizophrenia |
| 3-7 | SoftGel | Camelina | 420 | 10 | | | | 3 | | 500 | Pain; Nausea, |
| | | or krill | | | | | | | | | Inflammation, |
| | | | | | | | | | | | Appetite |
| | | | | | | | | | | | stimulation |
| 3-8 | SoftGel | Camelina | 420 | 10 | | | 10 | | | 500 | Pain; Anxiety; |
| | | or krill | | | | | | | | | Sleep, |
| | | | | | | | | | | | Inflammation |
| 3-9 | SoftGel | Camelina | 420 | 1 | | | 10 | | | 500 | Anti-epileptic, |
| | | or krill | | | | | | | | | Chronic Pain, |
| | | | | | | | | | | | Inflammation, |
| | | | | | | | | | | | Schizophrenia |
| 3-10 | SoftGel | Camelina | 420 | 1 | | | 10 | | | 500 | Anti-epileptic, |
| | | or krill | | | | | | | | | Chronic Pain, |
| | | | | | | | | | | | Inflammation, |
| | | | | | | | | | | | Schizophrenia |
| 3-11 | SoftGel | Camelina | 420 | | | 10 | 10 | | | 500 | Pain; Anxiety; |
| | | or krill | | | | | | | | | Sleep, |
| | | | | | | | | | | | Inflammation; |
| | | | | | | | | | | | Diabetes; Appetite |
| | | | | | | | | | | | suppression |
| 3-12 | SoftGel | Camelina | 420 | 1 | | | 20 | | | 500 | Anti-epileptic, |
| | | or krill | | | | | | | | | Chronic Pain, |
| | | | | | | | | | | | Inflammation, |
| | | | | | | | | | | | Schizophrenia |
| 3-13 | SoftGel | Camelina | 410 | 1 | | | 30 | | | 500 | Anti-epileptic, |
| | | or krill | | | | | | | | | Chronic Pain, |
| | | | | | | | | | | | Inflammation, |
| | | | | | | | | | | | Schizophrenia |
| 3-14 | SoftGel | Camelina | 400 | 1 | | | 40 | | | 500 | Anti-epileptic, |
| | | or krill | | | | | | | | | Chronic Pain, |
| | | | | | | | | | | | Inflammation, |
| | | | | | | | | | | | Schizophrenia |
| 3-15 | SoftGel | Camelina | 210 | 5 | | | | | | 250 | Pain, Appetite |
| | | or krill | | | | | | | | | enhancement, |
| | | | | | | | | | | | Inflammation; |
| 3-16 | SoftGel | Camelina | 210 | 5 | | | 10 | | | 250 | Pain, Appetite |
| | | or krill | | | | | | | | | enhancement, |
| | | | | | | | | | | | Inflammation; |
| 3-17 | SoftGel | Camelina | 210 | 5 | | | 25 | | | 250 | Pain, Appetite |
| | | or krill | | | | | | | | | enhancement, |
| | | | | | | | | | | | Inflammation; |
| 3-18 | SoftGel | Camelina | 180 | 10 | | | | 2 | 1 | 250 | Pain, |
| | | or krill | | | | | | | | | Inflammation, |
| | | | | | | | | | | | Gastrointestinal |
| | | | | | | | | | | | disorders, |
| | | | | | | | | | | | Neurodegenerative |
| | | | | | | | | | | | disorders |
| 3-19 | SoftGel | Camelina | 180 | 6 | | | | 3 | 3 | 250 | Pain, |
| | | or krill | | | | | | | | | Inflammation, |
| | | | | | | | | | | | Gastrointestinal |
| | | | | | | | | | | | disorders, |
| | | | | | | | | | | | Neurodegenerative |
| | | | | | | | | | | | disorders |
| 3-20 | SoftGel | Camelina | 180 | | | | 6 | 3 | 3 | 250 | Pain, |
| | | or krill | | | | | | | | | Inflammation, |
| | | | | | | | | | | | Gastrointestinal |
| | | | | | | | | | | | disorders, |
| | | | | | | | | | | | Neurodegenerative |
| | | | | | | | | | | | disorders |

*including capsule shell and all carrier, filler, stabilizer, and anti-oxidant, etc.

The compositions and methods described herein are illustrative and not intended to be limiting on the claims of the invention more particularly set out below.

What is claimed is:

1. A cannabinoid formulation for oral administration, comprising:
one or more cannabinoids selected from the group consisting of:
0.1-100 mg tetrahydrocannabinol (THC);
0.1-750 mg tetrahydrocannabinolic acid (THCA);
0.1-750 mg cannabidiol (CBD);
0.1-750 mg cannabidiolic acid (CBDA); and
0.1-750 mg cannabigerol (CBG);
0.1-750 mg cannabinchromene (CBC);
and
a lipid carrier comprising or consisting of camelina oil.

2. A cannabinoid formulation for oral administration, comprising:
one or more cannabinoids selected from the group consisting of:
0.1-100 mg tetrahydrocannabinol (THC);
0.1-750 mg tetrahydrocannabinolic acid (THCA);
0.1-750 mg cannabidiol (CBD);
0.1-750 mg cannabidiolic acid (CBDA);
0.2-750 mg cannabinchromene (CBC);
0.1-750 mg cannabigerol (CBG); and
a lipid carrier comprising one or more lipids selected from the group consisting of:
camelina oil;
a marine phospholipid;
krill oil;
fish oil;
chia seed oil;
flaxseed oil; and
an oil comprising an omega-3 to omega-6 ratio of about 1.0 or higher, 1.5 or higher, 2.0 or higher, or 2.2 or higher.

3. The cannabinoid formulation of claim 1 or 2, wherein one or more of the one or more cannabinoids is dissolved and/or suspended in the lipid carrier.

4. The cannabinoid formulation of any one of claims 1 to 3 in a unit dosage form selected from a pill, tablet, capsule, film, wafer, lollipop, lozenge, oil, tincture or syrup.

5. The cannabinoid formulation of claim 4, wherein the formulation is an orally disintegrating tablet, film, or wafer.

6. The cannabinoid formulation of claim 4, wherein the formulation is a pill or tablet and further comprises an enteric coating for containing the one or more cannabinoids and the lipid carrier.

7. The cannabinoid formulation of any one of claims 4 to 6, wherein the formulation is a pill, tablet, or capsule and further comprises an outer shell that is substantially opaque to one or both of ultraviolet and visible light.

8. The cannabinoid formulation of any one of claims 1-7 further comprising a further carrier oil.

9. The cannabinoid formulation of any one of claims 1-8, further comprising a stabilizer.

10. The cannabinoid formulation of any one of claims 1-9, wherein the lipid carrier is present in the form of an emulsion.

11. The cannabinoid formulation of claim 10, wherein the emulsion is a nanoemulsion.

12. The cannabinoid formulation of any one of claims 1 to 11, wherein upon administration of the formulation to a subject, a target bioavailability in the subject is achieved,

13. The cannabinoid formulation of any one of claims 1-12 wherein one or more of the cannabinoids is in the form of an organic solvent-based extract of cannabis.

14. The cannabinoid formulation of any one of claims 1-13, further comprising at least one further cannabinoid selected from the group consisting of CBGA and tetrahydrocannabivarin (THCV).

15. The cannabinoid formulation of any one of claims 1-14, comprising CBD in an amount between 10-50 mg.

16. The cannabinoid formulation of any one of claims 1-15, comprising 25 mg CBD.

17. The cannabinoid formulation of any one of claims 1-16 comprising 500 mg CBD.

18. The cannabinoid formulation of any one of claims 3-17, wherein the cannabinoid is evenly dispersed within at least a portion of the unit dosage form.

19. The cannabinoid formulation of any one of claims 3-18, wherein a signifier which signifies a cannabinoid dosage is associated directly with the unit dosage form by embossing, or by colour, pattern or shape feature.

20. The cannabinoid formulation of claim 19 wherein the signifier is adapted to be directly interpreted by a consumer and/or is a machine-readable code.

21. The cannabinoid formulation of any one of claims 3-20, wherein the unit dosage form is contained in an individual blister pack sealed in an inert gas atmosphere comprising little or no oxygen.

22. The cannabinoid formulation of any one of claims 3-21, wherein the lipid carrier is present in the form of a nanoemulsion comprising lipid particles having an average particle size of about 20-100, 20-200, 20-300, 20-400, 20-500, 20-600, 100-300, 200-500, or 100-600 nm.

23. The cannabinoid formulation of any one of claims 1-22, wherein the lipid carrier comprises medium-chain fatty acids (MCFAs) comprising or consisting of 12-16 carbon atoms or long-chain fatty acids (LCFAs) comprising or consisting of 13 or more carbon atoms.

24. The cannabinoid formulation of any one of claims 1-23, wherein the formulation further comprises a surfactant, and/or is formulated to provide an emulsion upon exposure to a user's gut.

25. The cannabinoid formulation of claim 24, wherein the surfactant is Labrasol™.

26. The cannabinoid formulation of claim 24 or 25, wherein the formulation comprises a defined dose or combination dose of cannabinoid(s) selected from the list consisting of (each cannabinoid milligram amount about or equal to):
a. 25 mg THCA and 2 mg THC;
b. 25 mg THC;
c. 1 mg THCA, 25 mg CBDA, and 2 mg CBD;
d. 1 mg THCA and 25 mg CBD;
e. 25 mg THCA, 2 mg THC, 25 mg CBDA, and 2 mg CBD;
f. 25 mg THCA, 2 mg THC, and 2 mg CBD;
g. 25 mg THC and 25 mg CBD;
h. 25 mg THC and 2 mg CBD;
i. 1 mg THC, 25 mg CBD; 25 mg CBG, and 25 mg CBC
j. 25 mg THC and 25 mg THCV;
k. 9 mg THCA and 1 mg THC;
l. 10 mg THC;
m. 9 mg THCA, 1 mg THC, 9 mg CBDA, and 1 mg CBD;
n. 9 mg THCA, 1 mg THC, and 10 mg CBD;
o. 10 mg THC and 1 mg CBD;
p. 10 mg THC and 10 mg THCV;
q. 600 mg THC;
r. 600 mg THCA and 50 mg THC;
s. 100 mg THC;
t. 600 mg CBDA;
u. 25 mg THCA, 600 mg CBDA, and 60 mg CBD;
v. 100 mg CBD;
w. 4 mg THC and 100 mg CBD;
x. 600 mg CBD;
y. 25 mg THC and 600 mg CBD;
z. 600 mg CBG;
aa. 300 mg THCA and 300 mg CBDA;
bb. 300 mg THCA, 30 mg THC, 300 mg CBDA, and 30 mg CBD;
cc. 300 mg THCA and 30 mg CBD;
dd. 300 mg THCA, 30 mg THC, and 300 mg CBD;
ee. 100 mg THC and 100 mg CBD;
ff. 100 mg THC and 30 mg CBD;
gg. 300 mg THC and 300 mg CBG;
hh. 300 mg THC and 300 mg CBC;
ii. 300 mg CBD and 300 mg CBG;
jj. 300 mg CBD and 300 mg CBC;
kk. 300 mg CBD, 300 mg CBG, and 300 mg CBC;
ll. 10 mg THC, 250 mg CBD, 250 mg CBG, and 250 mg CBC;
mm. 100 mg THC and 500 mg THCV;
nn. 300 mg CBD and 300 mg THCV;
oo. 100 mg CBD and 100 mg THCV;
pp. 1 mg THC and 9 mg THCA;
qq. 1 mg THC and 9 mg THCA;
rr. 10 mg THC;
ss. 10 mg THC and 9 mg CBD;
tt. 10 mg THC and 10 mg CBD;
uu. 1 mg THC and 25 mg CBD;
vv. 100 mg THC and 3 mg CBG;
ww. 1 mg THC and 10 mg CBD;
xx. 10 mg THCV and 10 mg CBD;
yy. 1 mg THC and 20 mg CBD;
zz. 1 mg THC and 30 mg CBD;
aaa. 1 mg THC and 40 mg CBD;
bbb. 5 mg THC;
ccc. 5 mg THC and 10 mg CBD;
ddd. 5 mg THC and 25 mg CBD;
eee. 10 mg THC, 2 mg CBG and 1 mg CBC;
fff. 6 mg THC, 3 mg CBG and 3 mg CBC; and
ggg. 6 mg CBD, 3 mg CBG, and 2 mg CBC.

27. The cannabinoid formulation of any one of claims 1-26 wherein the lipid carrier comprises or consists of camelina oil.

28. The cannabinoid formulation of claim 12, wherein the formulation comprises a first defined dose amount of a dosed cannabinoid, further wherein the administration is by oral administration, and wherein the target bioavailability is a bioavailability of THC or a THC liver metabolite which is greater than a baseline bioavailability, the baseline bioavailability being that achievable from oral administration to the subject of a control composition containing the first defined dose amount of the dosed cannabinoid in a carrier comprising at least 90%, or consisting of, sesame oil.

29. The cannabinoid formulation of claim 28, further wherein the dosed cannabinoid is THC and wherein the cannabinoid formulation comprises about 30-50% of the defined dose amount of THC in an emulsion.

30. The cannabinoid formulation of claim 28 or 29, wherein the target bioavailability is a THC or THC liver metabolite bioavailability in the subject that is at least 140%, as measured by subject Cmax and/or subject AUC, of the baseline bioavailability.

31. The cannabinoid formulation of claim 30, wherein the target bioavailability is a THC or THC liver metabolite bioavailability in the subject that is: (i) at least 108.5%, at least 146.9%, at least 159.6%, or at least 200%, as measured by subject Cmax, and/or (ii) at least 144.3%; at least 159.6%, at least 200%, or at least 252.1%, as measured by subject AUC, of the baseline bioavailability.

32. The cannabinoid formulation of any one of claims 28-31, wherein the target bioavailability is a bioavailability of: THC; 11-OH-THC; and/or THC—COOH.

33. The cannabinoid formulation of any one of claims 6-32, comprising an enteric coating for containing the one or more cannabinoids and the lipid carrier, wherein the enteric coating degrades, dissolves, or otherwise provides a release of the cannabinoid and the lipid carrier in an environment having a pH value of below about 5.5, at about 5.5 or greater, at about 6.0 to about 6.5, at about 6.5 to 7.5, or greater than about 7.5.

34. The cannabinoid formulation of any one of claims 1 to 33, wherein the one or more cannabinoids in present in a defined dose.

35. A method of administering one or more cannabinoids to a subject, comprising orally administering to the subject a formulation comprising one or more cannabinoids selected from the group consisting of:
0.1-100 mg tetrahydrocannabinol (THC);
0.1-750 mg tetrahydrocannabinolic acid (THCA);
0.1-750 mg cannabidiol (CBD);
0.1-750 mg cannabidiolic acid (CBDA);
0.1-750 mg cannabinchromene (CBC); and
0.1-750 mg cannabigerol (CBG); and
a lipid carrier comprising one or more lipids selected from the group consisting of:
camelina oil;
a marine phospholipid;
fish oil;
krill oil;
chia seed oil;
flaxseed oil; and
an oil comprising an omega-3 to omega-6 ratio of about 1.0 or higher, 1.5 or higher, 2.0 or higher, or 2.2 or higher.

36. A method of administering one or more cannabinoids to a subject, comprising orally administering to the subject a formulation comprising one or more cannabinoids selected from the group consisting of:
0.1-100 mg tetrahydrocannabinol (THC);
0.1-750 mg tetrahydrocannabinolic acid (THCA);
0.1-750 mg cannabidiol (CBD);
0.1-750 mg cannabidiolic acid (CBDA);
0.1-750 mg cannabinchromene (CBC); and
0.1-750 mg cannabigerol (CBG); and
a lipid carrier comprising or consisting of camelina oil.

37. A method of administering a therapeutically and/or psychotropically effective amount of one or more cannabinoids to a subject, comprising orally administering to the subject a cannabinoid formulation of any one of claims 1-33.

38. The method of any of claims 35 to 37 wherein the subject is in need of treatment for pain, inflammation, anxiety, depression, insomnia, sleep disorders, lack of energy, lack of alertness, weight gain, obesity, diabetes, metabolic syndrome, nausea (acute or anticipatory), epilepsy, spasticity, schizophrenia, bi-polar disorder, cancer and neoplasia, chronic pain, osteoarthritic pain, bacterial and/or fungal infection, fibromyalgia, appetite enhancement and/or appetite suppression.

39. A method of administering a therapeutically and/or psychotropically effective amount of one or more cannabinoids to a subject, the method comprising orally administering to the subject a cannabinoid formulation comprising:
one or more cannabinoids selected from the group consisting of:
0.1-100 mg tetrahydrocannabinol (THC);
0.1-750 mg tetrahydrocannabinolic acid (THCA);
0.1-750 mg cannabidiol (CBD);
0.1-750 mg cannabidiolic acid (CBDA);
0.1-750 mg cannabinchromene (CBC);
and
0.1-750 mg cannabigerol (CBG); and
a lipid carrier comprising one or more lipids selected from the group consisting of:
camelina oil;
a marine phospholipid;
krill oil;
fish oil;
chia seed oil;
flaxseed oil; and
an oil comprising an omega-3 to omega-6 ratio of about 1.0 or higher, 1.5 or higher, 2.0 or higher, or 2.2 or higher.

40. A method of administering a therapeutically and/or psychotropically effective amount of one or more cannabinoids to a subject, the method comprising orally administering to the subject a cannabinoid formulation comprising:
one or more cannabinoids selected from the group consisting of:
0.1-100 mg tetrahydrocannabinol (THC);
0.1-750 mg tetrahydrocannabinolic acid (THCA);
0.1-750 mg cannabidiol (CBD);
0.1-750 mg cannabidiolic acid (CBDA);
0.1-750 mg cannabinchromene (CBC); and
0.1-750 mg cannabigerol (CBG); and
a lipid carrier comprising or consisting of camelina oil.

41. The method of claim 39 or 40, wherein one or more of the one or more cannabinoids is dissolved and/or suspended in the lipid carrier.

42. The method of any one of claims 39 to 41, wherein the cannabis formulation is in a unit dosage form selected from a pill, tablet, capsule, film, wafer, lollipop, lozenge, oil, tincture or syrup.

43. The method of claim 42, wherein the formulation is an orally disintegrating tablet, film, or wafer.

44. The method of claim 43, wherein the formulation is a pill or tablet and further comprises an enteric coating for containing the one or more cannabinoids and the lipid carrier.

45. The method of any one of claims 42 to 44, wherein the formulation is a pill, tablet, or capsule and further comprises an outer shell that is substantially opaque to one or both of ultraviolet and visible light.

46. The method of any one of claims 39-45 wherein the formulation further comprises a further carrier oil.

47. The method of any one of claims 39-46, wherein the formulation further comprises a stabilizer.

48. The method of any one of claims 39-47, wherein the lipid carrier is present in the formulation in the form of an emulsion.

49. The method of claim 48, wherein the emulsion is a nanoemulsion.

50. The method of any one of claims 39 to 49, wherein upon administration of the cannabinoid formulation to a subject, a target bioavailability in the subject is achieved,

51. The method of any one of claims 39-50 wherein one or more of the cannabinoids in the formulation is in the form of an organic solvent-based extract of cannabis.

52. The method of any one of claims 39-51, further comprising at least one further cannabinoid selected from the group consisting of CBGA, and tetrahydrocannabivarin (THCV).

53. The method of any one of claims 39-52, comprising CBD in an amount between 10-50 mg.

54. The method of any one of claims 39-53, comprising 25 mg CBD.

55. The method of any one of claims 39-54 comprising 500 mg CBD.

56. The method of any one of claims 42-55, wherein the cannabinoid is evenly dispersed within at least a portion of the unit dosage form.

57. The method of any one of claims 42-56, wherein the formulation further comprises a signifier which signifies a cannabinoid dosage is associated directly with the unit dosage form by embossing, or by colour, pattern or shape feature.

58. The method of claim 57 wherein the signifier is adapted to be directly interpreted by a consumer and/or is a machine-readable code.

59. The method of any one of claims 42-58, wherein the unit dosage form is contained in an individual blister pack sealed in an inert gas atmosphere comprising little or no oxygen.

60. The method of any one of claims 42-59, wherein the lipid carrier is present in the formulation in the form of a nanoemulsion comprising lipid particles having an average particle size of about 20-100, 20-200, 20-300, 20-400, 20-500, 20-600, 100-300, 200-500, or 100-600 nm.

61. The method of any one of claims 39-60, wherein the lipid carrier comprises medium-chain fatty acids MCFAs comprising or consisting of 12-16 carbon atoms or long-chain fatty acids LCFAs comprising or consisting of 13 or more carbon atoms.

62. The method of any one of claims 39-61, wherein the formulation further comprises a surfactant.

63. The method of claim 62, wherein the formulation comprises the one or more cannabinoids an emulsion.

64. The method of claim 62 or 63, wherein the formulation comprises a defined dose or combination dose of cannabinoid(s) selected from the list consisting of (each cannabinoid milligram amount about or equal to):
a. 25 mg THCA and 2 mg THC;
b. 25 mg THC;
c. 1 mg THCA, 25 mg CBDA, and 2 mg CBD;
d. 1 m
e. g THCA and 25 mg CBD;
f. 25 mg THCA, 2 mg THC, 25 mg CBDA, and 2 mg CBD;
g. 25 mg THCA, 2 mg THC, and 2 mg CBD;
h. 25 mg THC and 25 mg CBD;
i. 25 mg THC and 2 mg CBD;
j. 1 mg THC, 25 mg CBD; 25 mg CBG, and 25 mg CBC;
k. 25 mg THC and 25 mg THCV;
l. 9 mg THCA and 1 mg THC;
m. 10 mg THC;
n. 9 mg THCA, 1 mg THC, 9 mg CBDA, and 1 mg CBD;
o. 9 mg THCA, 1 mg THC, and 10 mg CBD;
p. 10 mg THC and 1 mg CBD;
q. 10 mg THC and 10 mg THCV;
r. 600 mg THC;
s. 600 mg THCA and 50 mg THC;
t. 100 mg THC;
u. 600 mg CBDA;
v. 25 mg THCA, 600 mg CBDA, and 60 mg CBD;
w. 100 mg CBD;
x. 4 mg THC and 100 mg CBD;
y. 600 mg CBD;
z. 25 mg THC and 600 mg CBD;
aa. 600 mg CBG;
bb. 300 mg THCA and 300 mg CBDA;
cc. 300 mg THCA, 30 mg THC, 300 mg CBDA, and 30 mg CBD;
dd. 300 mg THCA and 30 mg CBD;
ee. 300 mg THCA, 30 mg THC, and 300 mg CBD;
ff. 100 mg THC and 100 mg CBD;
gg. 100 mg THC and 30 mg CBD;
hh. 300 mg THC and 300 mg CBG;
ii. 300 mg THC and 300 mg CBC;
jj. 300 mg CBD and 300 mg CBG;
kk. 300 mg CBD and 300 mg CBC;
ll. 300 mg CBD, 300 mg CBG, and 300 mg CBC;
mm. 10 mg THC, 250 mg CBD, 250 mg CBG, and 250 mg CBC;
nn. 100 mg THC and 500 mg THCV;
oo. 300 mg CBD and 300 mg THCV;
pp. 100 mg CBD and 100 mg THCV;
qq. 10 mg THC, 2 mg CBG and 1 mg CBC;
rr. 6 mg THC, 3 mg CBG and 3 mg CBC; and
ss. 6 mg CBD, 3 mg CBG, and 2 mg CBC

65. The method of any one of claims 39-64 wherein the lipid carrier comprises or consists of camelina oil.

66. The method of claim 65, wherein the formulation comprises a first defined dose amount of a dosed cannabinoid, further wherein the administration is by oral administration, and wherein the target bioavailability is a bioavailability of THC or a THC liver metabolite which is greater than a baseline bioavailability, the baseline bioavailability being that achievable from oral administration to the subject of a control composition containing the first defined dose amount of the dosed cannabinoid in a lipid carrier comprising at least 90% sesame oil.

67. The method of claim 66, further wherein the dosed cannabinoid is THC and wherein the cannabinoid formulation comprises about 30-50% of the defined dose amount of THC in an emulsion.

68. The method of claim 66 or 67, wherein the target bioavailability is a THC or THC liver metabolite bioavailability in the subject that is at least 140%, as measured by subject Cmax and/or subject AUC, of the baseline bioavailability.

69. The method of claim 68, wherein the target bioavailability is a THC or THC liver metabolite bioavailability in the subject that is: (i) at least 108.5%, at least 146.9%, at least 159.6%, or at least 200%, as measured by subject Cmax, and/or (ii) at least 144.3%; at least 159.6%, at least 200%, or at least 252.1%, as measured by subject AUC, of the baseline bioavailability.

70. The method of any one of claims 66-69, wherein the target bioavailability is a bioavailability of: THC; 11-OH-THC; and/or THC—COOH.

71. The method of any one of claims 44-70, comprising an enteric coating for containing the one or more cannabinoids and the lipid carrier, wherein the enteric coating degrades, dissolves, or otherwise provides a release of the cannabinoid and the lipid carrier in an environment having a pH value of below about 5.5, at about 5.5 or greater, at about 6.0 to about 6.5, at about 6.5 to 7.5, or greater than about 7.5.

72. The method of any one of claims 35-71, wherein the subject has a disease or disorder treatable by the administration of the one or more cannabinoids.

73. The method of claim 72, wherein the disease or disorder is selected from the group consisting of: pain, inflammation, anxiety, depression, insomnia, sleep disorders, lack of energy, lack of alertness, weight gain, obesity, diabetes, metabolic syndrome, nausea (acute or anticipatory), epilepsy, spasticity, schizophrenia, bi-polar disorder, cancer and neoplasia, chronic pain, osteoarthritic pain, bacterial and/or fungal infection, fibromyalgia, appetite enhancement, appetite suppression, and any combination thereof.

74. The method of any one of claims 43 to 73, wherein the one or more cannabinoids is present in a defined dose.

75. Use of the formulation of any one of claims 1-34 for treatment of a disease or disorder in the subject, wherein the disease or disorder is selected from the group consisting of pain, inflammation, anxiety, depression, insomnia, sleep disorders, lack of energy, lack of alertness, weight gain, obesity, diabetes, metabolic syndrome, nausea (acute or anticipatory), epilepsy, spasticity, schizophrenia, bi-polar disorder, cancer and neoplasia, chronic pain, osteoarthritic pain, bacterial and/or fungal infection, fibromyalgia, appetite enhancement, appetite suppression, and any combination thereof.
